SEQUENCE LISTING (3 sequences)

SEQ ID NO: 1 - protein, 275 aa, Canditatus Methanomethylophilus alvus (strain Mx1201Ca)
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNLYSVMRDLRDHTDGPVKIFEMGSCFRKESHSGMHLEEFTMLNLVDMGPRGDATEVLKNYISVVMKAAGLPDYDLVQEESDVYKETIDVEINGQEVCSAAVGPHYLDAAHDVHEPWSGAGFGLERLLTIREKYSTVKKGGASISYLNGAKIN

SEQ ID NO: 2 - protein, 275 aa, Artificial Sequence
  V168C and M129L substitutions in SEQ ID NO 1
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNLYSVLRDLRDHTDGPVKIFEMGSCFRKESHSGMHLEEFTMLNLCDMGPRGDATEVLKNYISVVMKAAGLPDYDLVQEESDVYKETIDVEINGQEVCSAAVGPHYLDAAHDVHEPWSGAGFGLERLLTIREKYSTVKKGGASISYLNGAKIN

SEQ ID NO: 3 - protein, 454 aa, Methanosarcina mazei
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYLRKLDRALPDPIKIFEIGPCYRKESDGKEHLEEFTMLNFCQMGSGCTRENLESIITDFLNHLGIDFKIVGDSCMVYGDTLDVMHGDLELSSAVVGPIPLDREWGIDKPWIGAGFGLERLLKVKHDFKNIKRAARSESYYNGISTNL

(19) United States
(12) Patent Application Publication
(10) Pub. No.: US 2023/0287383 A1
(43) Pub. Date: Sep. 14, 2023

(54) PYRROLYSYL-tRNA SYNTHETASE VARIANTS AND USES THEREOF

(71) Applicant: enGenes Biotech GmbH, Wien (AT)

(72) Inventor: Birgit Wiltschi, Graz (AT)

(21) Appl. No.: 18/013,849

(22) PCT Filed: Jul. 2, 2021
(86) PCT No.: PCT/EP2021/068279

§ 371 Date: Dec. 29, 2022

(30) Foreign Application Priority Data
Jul. 3, 2020 (LU) LU101905

(51) Int. Cl. C12N 9/00; C12N 15/63

(52) U.S. Cl. CPC C12N 9/93; C12N 15/63; C12Y 601/01026

(57) ABSTRACT

The present invention relates to the field of pyrrolysyl-tRNA synthetases, their variants and uses thereof. Particularly, the present invention relates to variants (mutants) of parent pyrrolysyl-tRNA synthetases, wherein said variants have pyrrolysyl-tRNA synthetase activity and exhibit altered properties relative to the corresponding parent pyrrolysyl-tRNA synthetase.

This application contains a Sequence Listing in a computer readable form, which is incorporated herein by reference.

### TECHNICAL FIELD

The present invention relates to the field of pyrrolysyl-tRNA synthetases (also referred to as pyrrolysine-tRNA ligases), their variants and uses thereof. The present invention further relates to variants (mutants) of parent pyrrolysyl-tRNA synthetases, wherein said variants have pyrrolysyl-tRNA synthetase activity and exhibit altered properties relative to a corresponding parent pyrrolysyl-tRNA synthetase.

### BACKGROUND OF THE INVENTION

With a few exceptions, all known organisms use the same set of 20 canonical amino acids (cAAs) prescribed by the genetic code for protein biosynthesis. As a result, the chemical diversity in proteins is confined to this small and defined set of cAAs (Wan et al., Biochimica et Biophysica Acta (BBA)—Proteins and Proteomics (2014), 1844 (6): 1059-1070). Proteins implement a remarkable range of functions, but often they do not offer the chemistries that are desirable for biotechnological applications. These limitations may be overcome using non-canonical amino acids (ncAAs), which are a very diverse group of compounds. Though naturally not participating in protein translation, ncAAs offer a broad spectrum of side chain chemistries and many of them occur in nature (Walsh et al., Angewandte Chemie Int Ed (2013), 52 (28): 7098-7124). Currently, there are several approaches available to incorporate these ncAAs into proteins to exploit their broad structural and functional repertoire.

Tools for the site-specific incorporation of ncAAs into target proteins are known in the art (Wang et al., Science (2001), 292 (5516): 498-500; Hohsaka T, Sisido M. 2002. Incorporation of non-natural amino acids into proteins. Current Opinion in Chemical Biology 6(6): 809-815.). To do so, an engineered aminoacyl-tRNA synthetase (aaRS) is needed that accepts exclusively the ncAA and charges it on its cognate tRNA. For example, a tRNA suppressing a stop codon, such as tRNA_{CUA} may be used. This methodology is known as stop codon suppression (SCS) (Young et al., J Biol Chem (2010), 285 (15): 11039-11044). For SCS it is important that the pair consisting of the ncAA-specific aaRS and the tRNA_{CUA} is orthogonal (o-pair), which means it does not interfere with the endogenous translation system of the host (Wang (2001), loc. cit.). In early years, many of the o-pairs developed were based on the archaeal TyrRS/tRNA_{CUA}^{Tyr} from Methanocaldococus janaschii (Mj) (Ryu et al., Nature Methods (2006), 3 (4): 263-265). The Mj o-pairs have been vastly used for the incorporation of a whole set of different ncAAs (Dumas et al., Chemical Science (2015), 6 (1): 50-59).

A very efficient naturally occurring system for the reassignment of the TAG codon was first discovered in the archeaeal Methanosarcinaceae species. These archaea incorporate the lysine analog pyrrolysine (Pyl) into several methylamine methyltransferases in response to an in-frame TAG codon when facing certain environmental conditions (Srinivasan et al., Science (2002), 296 (5572): 1459-1462). The decoding of Pyl in Methanosarcinaceae is carried out by pyrrolysyl-tRNA synthetase (PyIRS) that specifically charges Pyl onto its cognate tRNA_{CUA}^{Pyl}. The charged Pyl-tRNA_{CUA}^{Pyl} is capable of suppressing an in-frame TAG codon by the incorporation of Pyl at that position (Krzycki et al., Curr Op Microbiol (2005), 8 (6): 706-712).

Since the discovery of the Pyl decoding machinery in Methanosarcinaceae species, Pyl containing proteins were also found in the gram-positive bacterium Desulfitobacterium hafniense, in the human intestinal bacterium Bilophila wadsworthia, as well as selected Clostridium and Deltabacteria species (Gaston et al., Curr Opin Microbiol (2011), 14 (3): 342-349). Even though several natural Pyl decoding machineries were identified, only a few of them have been established for site-specific modification of proteins applying the SCS approach. Among these are the highly homologous PyIRS/tRNA_{CUA} o-pairs from Methanosarcina mazei (Mm) (Nozawa et al., Nature (2009), 457 (7233): 1163-1167) and Methanosarcina barkeri (Mb) (Polycarpo et al., FEBS Letters (2006), 580 (28-29): 6695-6700). An invaluable feature of the PylRSs from Mm and Mb for protein engineering by SCS is the natural substrate promiscuity of both enzymes for a stunning set of Pyl derivatives. To understand the molecular function of the enzyme (Yanagisawa et al., Chem & Biol (2008), 15 (11): 1187-1197), the crystal structure of the catalytic domain of MmPyIRS was solved (Kavran et al., PNAS USA (2007), 104 (27): 11268-11273). The crystal structure facilitated the engineering of PylRSs for new substrates. In this way, the substrate scope of MmPyIRS and MbPyIRS was extended to other Pyl and lysine analogs (Wan (2014), loc. cit.). Furthermore, MmPyIRS was engineered to accept non-canonical aromatic amino acids (Wang et al., Mol Biosyst (2011), 7 (3): 714-717).

Meanwhile, MmPyIRS and MbPyIRS can be used for the site-specific incorporation of a palette of ncAAs in E. coli. Moreover, the archaeal PyIRS/tRNA_{CUA} pairs are orthogonal not only in E. coli but also in other organisms, such as yeasts (Hancock et al., J Am Chem Soc (2010), 132 (42): 14819-14824) and mammalian cells (Mukai et al., Biochem Biophys Res Comm (2008), 371 (4): 818-822). Besides the o-pairs from Mm and Mb, a proof of concept on the successful exploitation of the bacterial PyIRS/tRNA_{CUA}^{Pyl} from Desulfitobacterium hafniense for site-specific incorporation of several Pyl analogs into target proteins in E. coli were published (Herring et al., Nucleic Acid Res (2007), 35 (4): 1270-1278; Katayama et al., Biosci Biotechnol Biochem (2012), 76 (1): 205-208). However, a broadly applicable orthogonal DhPyIRS/DhtRNA_{CUA} pair has not yet been reported.

O-pairs derived from Pyl decoding machineries provide a useful tool for the site-specific modification of proteins by SCS. Even though the Mm and Mb o-pairs are well established, more o-pairs could be recruited for their application in SCS. Alternative or more advantageous aminoacyl-tRNA synthetases, alone or as part of o-pairs might broaden the toolbox for SCS and may spark improvements with regard to efficiencies or substrate scopes.

The wildtype MmPyIRS/MmtRNA_{CUA}^{Pyl} (MmOP) has been used for the incorporation of AzK. The wildtype OTS MaPyIRS/MatRNA_{CUA}^{Pyl} (MaOP) can incorporate AzK, albeit at a much lower efficiency than the benchmark MmOP.

### SUMMARY OF THE INVENTION

The present invention relates to a variant of a parent aminoacyl-tRNA synthetase (e.g., EC:6.1.1.-.), wherein said variant comprises substitutions at positions corresponding to positions 168 and 129 of the amino acid sequence set forth in SEQ ID NO: 1 (e.g., MaPyIRS), (e.g., using the numbering of SEQ ID NO: 1), wherein said variant comprises at least the following combination of substitutions: X168C+X129L (e.g., V168C+M129L), wherein said variant is a polypeptide having at least 70%, e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, but less than 100% sequence identity with the amino acid sequence set forth in SEQ ID NO: 1 (e.g., MaPyIRS), wherein said variant has aminoacyl-tRNA synthetase activity (e.g., EC:6.1.1.-.). The present invention further relates to a variant MaPyIRS having M129L+V168C substitutions (SEQ ID NO: 2) that can efficiently recognize AzK.

The present application satisfies this demand by the provision of the variants described herein below, characterized in the claims and illustrated by the appended Examples and Figures.

Overview of the Sequence Listing

As described herein references are made to UniProtKB Accession Numbers (http://www.uniprot.org/, e.g., as available in UniProtKB Release 2020_01 published Feb. 26, 2020).

SEQ ID NO: 1 is the amino acid sequence of an exemplary parent pyrrolysyl-tRNA synthetase of the present invention derived from Canditatus Methanomethylophilus alvus (strain Mx1201Ca), which may also be referred to as “MmaPyIRS” or “MaPyIRS” herein, e.g., UniProtKB Accession Number: M9SC49.

SEQ ID NO: 2 is the amino acid sequence of the variant of the parent pyrrolysyl-tRNA synthetase having SEQ ID NO: 1 having V168C and M129L substitutions.

SEQ ID NO: 3 is the amino acid sequence of a pyrrolysyl-tRNA synthetase from Methanosarcina mazei, which may be referred to as “MmPyIRS” herein, e.g., UniProtKB Accession Number: Q8PWY1.

### BRIEF DESCRIPTION OF THE DRAWINGS

FIG. 1: The MaPyIRS/MatRNA_{CUA}^{Pyl} wildtype pair does not efficiently incorporate AzK. The incorporation of BocK, AllocK and AzK by the MmPyIRS/MmtRNA_{CUA}^{Pyl} and MaPyIRS/MatRNA_{CUA}^{Pyl} o-pairs was compared. The ncAAs were supplemented in the medium at 5 mM. eGFP fluorescence (λₑₓ 488 nm; λₑₘ 508 nm) was assessed after 4 hours (white bars) and overnight (black bars) induction. Fluorescence units were correlated to cell density (F_{488;508}/D₆₀₀) and the average of eight technical replicates is shown; the error bars indicate the standard deviation.

FIG. 2: The MaPyIRS M129L V168C/MatRNA_{CUA}^{Pyl} mutant o-pair shows substantially improved AzK incorporation. The incorporation of AzK by the MmPyIRS/MmtRNA_{CUA}^{Pyl} and the MaPyIRS/MatRNA_{CUA}^{Pyl} wild type and mutant o-pairs was compared. The ncAA was supplemented in the medium at 5 mM. eGFP fluorescence (λₑₓ 485 nm; λₑₘ 530 nm) was assessed after overnight induction. eGFP wild type was expressed from the same plasmid carrying the MmOP. Fluorescence units were correlated to cell density (F_{485;530}/D₆₀₀) and the average of at least two technical replicates is shown; the error bars indicate the standard deviation.

FIG. 3: SDS-PAGE analysis of the expression of eGFP Y40am in the presence of AzK with the MaPyIRS M129L V168C/MatRNA_{CUA}^{Pyl} mutant o-pair in comparison to MmOP and MaOP corroborates the fluorescence analysis. eGFP expression was assessed after overnight induction, two expressions of eGFP Y40AzK with the mutant MaOP are shown. eGFP wild type was expressed from the same plasmid carrying the MmOP. Samples of corresponding cell density were analyzed. The calculated molecular weight of eGFP is 28 kDa. M, molecular weight marker.

FIG. 4: The MaPyIRS M129L V168C/MatRNA_{CUA}^{Pyl} mutant o-pair produces more target protein than MmOP with less or the same amount of AzK. MaOP LC, MaPyIRS M129L V168C/MatRNA_{CUA}^{Pyl}; MmOP, PyIRS and RNA_{CUA}^{Pyl} from M. mazel, both o-pairs were used in BL21(DE3) cells; UNINDUCED, eGFP Y40am expression was not induced; INDUCED, expression was induced with 1 mM IPTG. Different concentrations of AzK were provided in the medium either 2 hours before induction with IPTG, or in parallel with IPTG. eGFP fluorescence (λₑₓ 485 nm; λₑₘ 530 nm) was assessed after overnight induction and was normalized to cell density (D₆₀₀). The average of at least 3 replicates is shown and the error bars indicate the standard deviation.

### DETAILED DESCRIPTION OF THE INVENTION

There is a need for improved orthogonal ncAA translation systems (OTSs) with greater functionality in the field of protein expression, e.g., (i) to site-specifically label proteins of choice with ncAAs carrying, for instance, reactive bioorthogonal groups for site-specific attachment of ligands such as small molecules or biomolecules; and/or (ii) to tune protein function, e.g. enzyme activity.

The present invention relates to the use of the wildtype MaPyIRS/MatRNA_{CUA}^{Pyl} pair (MaOP) for the incorporation of different lysine derivatives, such as N^{ε}-t-butyloxycarbonyl-L-lysine (BocK), N^{ε}-allyloxycarbonyl-L-lysine (AllocK), N^{ε}-(prop-2-ynyloxycarbonyl)-L-lysine (AlkyneK) and N^{ε}-((2-azidoethoxy)carbonyl)-L-lysine (AzK) (e.g., WO 2018/185222 A1). AzK is a very important lysine derivative as its azido-group in the side chain can be exploited for bioorthogonal coupling reactions, e.g. by Cu(I) catalyzed or strain-promoted [1+3] cycloadditions between azide and alkyne groups. MaOP can incorporate AzK, however, less efficiently than the broadly applied wildtype MmPyIRS/MmtRNA_{CUA}^{Pyl} o-pair (MmOP), which represents the benchmark. Accordingly, there is a need for engineered variants of MaPyIRS that can become equally efficient in the incorporation of AzK as MmPyIRS in order to generate OTS for AzK.

Accordingly, the technical problem underlying the present invention may be formulated as to comply with the needs set out above. The technical problem has been solved by means and methods as described herein as defined in the claims.

The present invention provides mutants (e.g., variants) of the pyrrolysyl-tRNA synthetase of Methanomethylophilius alvus (MaPyIRS), which accept non-canonical substrates, particularly non-canonical amino acids (ncAAs) with reactive bioorthogonal groups in the side chain, such as an azido-group. The mutants can, for example, form part of so-called orthogonal translation systems consisting of the mutant synthetase and a corresponding amber suppressor tRNA (orthogonal pair, o-pair, OP) for the site-specific incorporation of said ncAAs, e.g., into proteins in response to an amber stop codon.

The present invention addresses the technical problem inter alia by comparing the structures of the two enzymes and identifying six residues in the amino acid binding pocket of MmPyIRS that are important for the interaction of the enzyme with its amino acid substrates. Four out of the six residues were found to be identical: MaPyIRS Y126, N166, Y206, W239 corresponding to MmPyIRS Y306, N346, Y384, W417. The numbers differ in the two enzymes because MmPyIRS contains an N-terminal extension that is missing in MaPyIRS. While positions 129 in MaPyIRS is occupied by M, the corresponding position 309 in MmPyIRS is L. Similarly, the corresponding positions MaPyIRS V168 and MmPyIRS C348 differ.

In the course of the present invention mutant MaPyIRS M129L+V168C (SEQ ID NO: 2) was generated that was able to incorporate AzK more efficiently than the parent wildtype enzyme (i.e. mutant has an improved property). It was surprising how efficiently MaPyIRS M129L V168C can incorporate AzK: it is at least as efficient as the MmPyIRS wildtype. In parallel, its ability to incorporate other lysine derivatives was not significantly changed. This means that this new mutant combines the best properties of its both wildtype parent proteins (e.g., SEQ ID NOs: 1 and 3).

In some aspects the present invention relates to an OTS consisting of MaPyIRS/MatRNA_{CUA}^{Pyl} to incorporate the reactive ncAA N-epsilon-((2-azidoethoxy)carbonyl)-L-lysine (AzK) into the anti-HER2-specific affibody Z_{HER2:2891}-HER2 is a receptor protein which is particularly abundant on the surface of breast cancer cells, and Z_{HER2:2891} can be used to target breast cancer cells, e.g. for drug delivery or imaging.

The mutant OTS of the present invention incorporated the ncAA into the affibody very efficiently, at least equally efficiently as the benchmark OTS MmPyIRS/MmtRNA_{CUA}^{Pyl}. To demonstrate the general applicability of the OTS of the present invention, the mutant OTS MaPyIRS/MatRNA_{CUA}^{Pyl} was used to site-specifically label green fluorescent protein with AzK. The mutant OTS of the present invention was found to be at least as efficient as the benchmark OTC. The incorporation of AzK was confirmed by mass analysis.

### Definitions

As referred herein “EC numbers” (Enzyme Commission numbers) may be used to refer to enzymatic activity according to the Enzyme nomenclature database, Release of Feb. 26, 2020 (e.g., available at https://enzyme.expasy.org/). The EC number refers to Enzyme Nomenclature 1992 from NC-IUBMB, Academic Press, San Diego, Calif., including supplements 1-5 published in Eur. J. Biochem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively.

As referred herein “aminoacyl-tRNA synthetase” (e.g., EC:6.1.1.-.) includes the following enzymatic activities (EC numbers): 6.1.1.1 (Tyrosine—tRNA ligase); 6.1.1.2 (Tryptophan—tRNA ligase); 6.1.1.3 (Threonine—tRNA ligase); 6.1.1.4 (Leucine—tRNA ligase); 6.1.1.5 (Isoleucine—tRNA ligase); 6.1.1.6 (Lysine—tRNA ligase); 6.1.1.7 (Alanine—tRNA ligase); 6.1.1.9 (Valine—tRNA ligase); 6.1.1.10 (Methionine—tRNA ligase); 6.1.1.11 (Serine—tRNA ligase); 6.1.1.12 (Aspartate—tRNA ligase); 6.1.1.13 (D-alanine—poly(phosphoribitol) ligase); 6.1.1.14 (Glycine—tRNA ligase); 6.1.1.15 (Proline—tRNA ligase); 6.1.1.16 (Cysteine—tRNA ligase); 6.1.1.17 (Glutamate—tRNA ligase); 6.1.1.18 (Glutamine—tRNA ligase); 6.1.1.19 (Arginine—tRNA ligase); 6.1.1.20 (Phenylalanine—tRNA ligase); 6.1.1.21 (Histidine—tRNA ligase); 6.1.1.22 (Asparagine—tRNA ligase); 6.1.1.23 (Aspartate—tRNA(Asn) ligase); 6.1.1.24 (Glutamate—tRNA(Gln) ligase); 6.1.1.26 (Pyrrolysine—tRNA(Pyl) ligase); 6.1.1.27 (O-phosphoserine—tRNA ligase).

As referred herein “pyrrolysyl-tRNA synthetase activity” (e.g., EC:6.1.1.26) includes the following enzymatic activity: ATP+L-pyrrolysine+tRNA^{(Pyl)}=>AMP+diphosphate+L-pyrrolysyl-tRNA^{(Pyl)}.

As used herein, the term “corresponding to” may refer to a way of determining the specific amino acid of a sequence wherein reference is made to a specific amino acid sequence (e.g., US2020071638). E.g. for the purposes of the present invention, when references are made to specific amino acid positions, the skilled person would be able to align another amino acid sequence to said amino acid sequence that reference has been made to, in order to determine which specific amino acid may be of interest in said another amino acid sequence. Alignment of another amino acid sequence with e.g. the sequence as set forth in SEQ ID NOs: 1, 2, or 3, or any other sequence listed herein, has been described elsewhere herein. Alternative alignment methods may be used, and are well-known for the skilled person.

As used herein, the term “improved property”, refers to a characteristic associated with a variant that is improved compared to the parent. Such improved properties may include, but are not limited to, substrate specificity and/or enzymatic efficiency and/or stability etc.

Parent or parent aminoacyl-tRNA synthetase: The term “parent” or “parent aminoacyl-tRNA synthetase” as used herein, may refer to an aminoacyl-tRNA synthetase to which an alteration (e.g., substitution/s) is made to produce the enzyme variants of the present invention, e.g., may refer to the parent aminoacyl-tRNA synthetase of SEQ ID NO: 1, 2, or 3, or any other aminoacyl-tRNA synthetase (e.g., EC:6.1.1.-.) having at least 70% sequence identity to any of the polypeptides of SEQ ID NOs: 1, 2, or 3. The parent aminoacyl-tRNA synthetase may also be a polypeptide comprising a fragment of SEQ ID NOs: 1, 2, or 3, i.e. the parent aminoacyl-tRNA synthetase may be a fusion polypeptide having aminoacyl-tRNA synthetase activity as defined elsewhere herein.

Sequence identity: The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used may be gap open penalty of 10, gap extension penalty of 0.5, and the EBLOSUM62 (EMBOSS version of BLOSUM62) substitution matrix. The output of Needle labeled “longest identity” (obtained using the no-brief option) is used as the percent identity and is calculated as follows:

(Identical Residues×100)/(Length of Alignment−Total Number of Gaps in Alignment).

Alternatively, the parameters used may be gap open penalty of 10, gap extension penalty of 0.5, and the EDNAFULL (EMBOSS version of NCBI NUC4.4) substitution matrix. The output of Needle labeled “longest identity” (obtained using the no-brief option) is used as the percent identity and is calculated as follows:

(Identical Deoxyribonucleotides×100)/(Length of Alignment−Total Number of Gaps in Alignment).

Variant: The term “variant” as used herein, may refer to a polypeptide having aminoacyl-tRNA synthetase activity comprising a mutation, i.e., a substitution, insertion, and/or deletion, at one or more (e.g., several) positions relative to the “parent” aminoacyl-tRNA synthetase, e.g., having SEQ ID NO: 1. A substitution means replacement of the amino acid occupying a position with a different amino acid; a deletion means removal of the amino acid occupying a position; and an insertion means adding an amino acid adjacent to and immediately following the amino acid occupying a position. The variants of the present invention have at least 20%, e.g., at least 40%, at least 50%, at least 60%, at least 70%, at least 80%, at least 90%, at least 95%, or at least 100% of the aminoacyl-tRNA synthetase activity of SEQ ID NOs: 1, 2, or 3.

Wild-type aminoacyl-tRNA synthetase: The term “wild-type aminoacyl-tRNA synthetase” as used herein may refer to an aminoacyl-tRNA synthetase expressed by a naturally occurring microorganism, such as a bacterium, yeast, or filamentous fungus found in nature.

Conventions for Designation of Variants:

The variant, i.e. mutated, amino acids in the polypeptides of the invention are defined by reference to the amino acid numbering of SEQ ID NO: 1 (e.g., using the numbering of SEQ ID NO: 1).

For the purposes of the present invention, the polypeptide disclosed in SEQ ID NO: 1 is used to determine the corresponding amino acid residue in another aminoacyl-tRNA synthetase polypeptide. However, the skilled person would recognize that the sequence of SEQ ID NO: 2 may also be used to determine the corresponding amino acid residue in another aminoacyl-tRNA synthetase polypeptide. The amino acid sequence of another aminoacyl-tRNA synthetase is aligned with the polypeptide disclosed in SEQ ID NO: 1, and based on the alignment, the amino acid position number corresponding to any amino acid residue in the polypeptide disclosed in SEQ ID NO: 1 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

Identification of the corresponding amino acid residue in another aminoacyl-tRNA synthetase can be determined by an alignment of multiple polypeptide sequences using several computer programs including, but not limited to, MUSCLE (multiple sequence comparison by log-expectation; version 3.5 or later; Edgar, Nucleic Acids Res (2004), 32 (5): 1792-1797 Edgar, Nucleic Acids Res (2004), 32 (5): 1792-1797), MAFFT (version 6.857 or later; Katoh et al., Nucleic Acids Res (2002), 30 (14): 3059-3066; Katoh et al., Nucleic Acids Res (2005), 33 (2): 511-518; Katoh et al., Bioinformatics (2007), 23 (3): 372-374; Katoh et al., Methods Mol Biol (2009), 537: 39-64; Katoh et al., Bioinformatics (2010), 26 (15): 1899-1900), and EMBOSS EMMA employing ClustalW (1.83 or later; Thompson et al., Nucleic Acids Res (1994), 22 (22): 4673-4680), using their respective default parameters.

When the other aminoacyl-tRNA synthetase has diverged from the polypeptide of SEQ ID NO: 1 such that traditional sequence-based comparison fails to detect their relationship (Lindahl et al., J Mol Biol (2000), 295 (3): 613-625), other pairwise sequence comparison algorithms can be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Altschul et al., Nucleic Acids Res (1997), 25 (17): 3389-3402). Even greater sensitivity can be achieved if the family or superfamily for the polypeptide has one or more representatives in the protein structure databases. Programs such as GenTHREADER (Jones, J Mol Biol (1999), 287 (4): 797-815; McGuffin et al., Bioinformatics (2003), 19 (7): 874-881) utilize information from a variety of sources (PSI-BLAST, secondary structure prediction, structural alignment profiles, and solvation potentials) as input to a neural network that predicts the structural fold for a query sequence. Similarly, the method of Gough et al., J Mol Biol (2001), 313 (4): 903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example, the SCOP super-families of proteins have been structurally aligned, and those alignments are accessible and downloadable. Two or more protein structures can be aligned using a variety of algorithms such as the distance alignment matrix (Holm et al., Proteins (1998), 33 (1): 88-96) or combinatorial extension (Shindyalov et al., Protein Eng (1998), 11 (9): 739-747), and implementation of these algorithms can additionally be utilized to query structure databases with a structure of interest in order to discover possible structural homologs (e.g., Holm et al., Bioinformatics (2000), 16 (6): 566-567).

In describing the aminoacyl-tRNA synthetase variants of the present invention, the nomenclature described below is adapted for ease of reference. The accepted IUPAC single letter or three letter amino acid abbreviations are employed.

Substitutions. For an amino acid substitution, the following nomenclature is used: Original amino acid, position, substituted amino acid. Accordingly, the substitution of e.g. M at position 129 with L is designated as “M129L”. Multiple mutations are separated by addition marks (“+”), e.g., “V168C+M129L”, representing substitutions at positions 168 and 129 of V with C and M with L, respectively. Substitutions may also be indicated with an “X” preceding a position number, which means that any original amino acid in a parent aminoacyl-tRNA synthetase, e.g., other than the aminoacyl-tRNA synthetase of SEQ ID NO: 2, may be substituted at the corresponding indicated position in the parent aminoacyl-tRNA synthetase. For example, “X168C” means that any amino acid residue at position 168 of a parent aminoacyl-tRNA synthetase other than C is substituted with C.

Multiple alterations. Variants comprising multiple alterations are separated by addition marks (“+”), e.g., “V168C+M129L” representing a substitution of V and M at positions 168 and 129 with C and L, respectively.

Numbering of Amino Acid Positions/Residues. If nothing else is mentioned the amino acid numbering used herein correspond to that of the SEQ ID NO: 1.

In the context of the present invention aminoacyl-tRNA synthetases (also referred to herein as “aaRS”) as described herein, e.g. those for pyrrolysine (pyrrolysyl-tRNA synthetases, also referred to herein as “PyIRS”), particularly from Candidatus Methanomethylophilus alvus (Mma or Ma) exhibit highly efficient capability to introduce aliphatic amino acids, particularly lysine derivatives, into polypeptides during translation. In context with the present invention, it is also envisaged that aminoacyl-tRNA synthetases and their variants as described herein are further or alternatively capable of introducing aromatic amino acids into polypeptides during translation.

As generally known in the art, aminoacyl-tRNA synthetases (aaRS) are capable of attaching amino acids onto tRNAs cognate to the respective aaRS. As known in the art and in context with the present invention, “cognate tRNA” may mean that such tRNA is usually specifically recognized or preferred by the respective corresponding aaRS. This attachment step corresponds to an esterification step which is catalyzed by the aaRS, so that the respective tRNA is charged (or aminoacylated, attached, linked, loaded, etc.) with the respective amino acid to form an aminoacyl-tRNA. Accordingly, as used herein, this attachment step may also be referred to as charging, loading, esterification, aminoacylation, or the like as understood by the person of skill in the art. tRNAs comprise an anticodon which are capable of identifying corresponding codons on the mRNA, usually either specific/base-by-base, or by wobble base pairing as known in the art. Also, as known in the art, usually tRNAs are specific for or prefer a particular amino acid with which the cognate tRNA may be charged by a corresponding aaRS.

In one embodiment of the present invention, the amino acids which are charged onto the cognate tRNA are non-canonical.

“Aliphatic” amino acids as used herein comprise non-aromatic amino acids, e.g., canonical aliphatic amino acids such as isoleucine, leucine, methionine, valine, alanine, glycine, and proline (particularly isoleucine, leucine, methionine, and valine), as well as hydroxyl-, sulfur- and amide-containing amino acids, e.g., serine, threonine, asparagine, glutamine, cysteine as well as basic amino acids, e.g., arginine, lysine and pyrrolysine, as well as acidic amino acids, e.g., aspartic acid and glutamic acid, as well as non-canonical amino acids such as ornithine and derivatives, norleucine, methoxinine, S-allyl-L-homocysteine, S-propargyl-L-homocysteine, L-azidohomoalanine, O-allyl-L-homocysteine, O-propargyl-L-homocysteine, S-azidopropyl-L-homocysteine, alanine derivatives carrying saturated and unsaturated C7-C10 side chains, or beta-hydroxy amino acids such as 2-amino-3-(bicyclo[2.2.1]hept-5-en-2-yl)-3-hydroxypropanoic acid.

“Aromatic” amino acids as used herein comprise an aromatic ring (e.g., phenyl, hydroxyphenyl, imidazole or indole side chain), e.g., canonical aromatic amino acids such as histidine, phenylalanine, tryptophan, and tyrosine, as well as non-canonical amino acids such as S-furyl-L-homocysteine, para-azido-L-phenylalanine, para-acetyl-L-phenylalanine, or para-propargyloxy-L-phenylalanine.

In one embodiment of the present invention, the inventive variants of aminoacyl-tRNA synthetases as described and provided herein allow aminoacylation of cognate tRNAs having an anticodon matching to a stop codon, preferably amber (UAG), ochre (UAA) or opal (UGA). According to the present invention, for example, the tRNA cognate to Pyl may be charged with Pyl by the enzyme PyIRS (which catalyzes aminoacylation of tRNA^{Pyl} with Pyl) according to the present invention. The tRNA^{Pyl} has the anticodon CUA (tRNA_{CUA}), which matches to the amber stop codon UAG. That is, Pyl is encoded by the amber stop codon UAG. In this context, in one embodiment of the present invention, the tRNA corresponding to an aliphatic amino acid which are aminoacylated by the aaRS (e.g., PyIRS) encoded by a polynucleotide as described and provided herein may be tRNA^{Pyl} (tRNA_{CUA}) (may also be referred to as tRNA^{Pyl}_{CUA}), and the corresponding aliphatic amino acid may be Pyl or another lysine derivative.

In context with the present invention, by expressing a variant of aminoacyl-tRNA synthetase (e.g., PyIRS variant) as described and provided herein, preferably together with its orthogonal tRNA (e.g., tRNA^{Pyl} (tRNA_{CUA})) recognizing a stop codon (e.g., amber, ochre or opal; preferably amber) or any sense codon, it is possible to suppress such stop codons as the translation does not stop at the stop codon but the amino acid (e.g., Pyl) corresponding to its cognate tRNA (e.g., tRNA^{Pyl} (tRNA_{CUA})) is introduced into the polypeptide during the translation process. Analogously, for sense codons, it may be possible to introduce a non-canonical amino acid at that site instead of a canonical which is usually encoded by such codon.

Generally, as used herein, the term “catalyzing aminoacylation of (an) amino acid(s) (with its corresponding/cognate tRNA)” means the process of adding an aminoacyl group to a compound by covalently linking (charging, attaching, loading, linking, bonding, or the like) the respective amino acid to the 3′ end of the corresponding/cognate tRNA molecule to form an aminoacyl-tRNA as known in the art and also shown and exemplified herein. Methods for assessing the capability of a given enzyme (aaRS) to catalyze such aminoacylation steps are known in the art and are described, e.g., in Francklyn et al., Methods (2008), Methods for kinetic and thermodynamic analysis of aminoacyl-tRNA synthetases, 44 (2), 100-118. It is also possible to indirectly or implicitly show successful catalysis of aminoacylation by a given enzyme (aaRS), based on the observation of introduction of a given amino acid (e.g., Pyl) into a polypeptide as this cannot take place without prior aminoacylation of the cognate tRNA with the corresponding amino acid. Also comparisons with negative control samples, e.g., without addition of the tRNA cognate to the respective enzyme (aaRS), allow indirect or implicit conclusion of successful aminoacylation catalysis ability of the enzyme (aaRS) as readily understood by the person skilled in the art. For example, in context with the present invention, a given enzyme (aaRS) may be considered to exhibit aminoacylation ability (i.e. ability to catalyze aminoacylation) if introduction (or incorporation, herein used synonymously in this context) of the corresponding amino acid into a polypeptide can be observed. Introduction or incorporation of an amino acid into a polypeptide may be observed by any method known in the art, comprising inter alia mass spectrometry or Edman degradation as known in the art and also described and exemplified herein. In context with the present invention, this aminoacylation process may preferably be catalyzed by polypeptides acting as aminoacyl-tRNA synthetases as described and provided herein and encoded by the polynucleotides as described and provided herein. Said term may also be referred to herein as esterification of the cognate tRNA with an amino acid. The capability of polypeptides encoded by polynucleotides as described and provided herein to catalyze aminoacylation of cognate tRNAs with the respective amino acids may also be referred to herein as introducing or incorporating such amino acids into a polypeptide, which usually takes place during the translation process of polypeptide synthesis. The variants of aminoacyl-tRNA synthetases according to the present invention are capable of catalyzing the aminoacylation of its cognate tRNA with an aliphatic or aromatic amino acid to form an aminoacyl-tRNA.

The term “cognate tRNA” as used in context with the present invention preferably means the tRNA which is charged or bonded with its corresponding amino acid to form an aminoacyl-tRNA (also referred to herein as “aa-tRNA”), a step which is preferably catalyzed by polypeptides encoded by polynucleotides as described and provided herein. “Cognate” in this sense is understood by the person skilled in the art and may particularly mean a pair of aaRS and tRNA carrying the corresponding anticodon corresponding to the codon for said amino acid on the mRNA, either base-by-base or by wobble base pairing as known in the art. That is, a given aaRS recognizes a specific cognate tRNA and loads (charges, etc. as described herein) it with a corresponding amino acid. For example, a tRNA_{GAA} (GAA being the anticodon of the tRNA) is charged with the corresponding amino acid phenylalanine (Phe) which is encoded on the mRNA by the codon UUC (or UUU). The tRNA_{GAA} is recognized by the specific aminoacyl-tRNA synthetase for phenylalanine, phenylalanyl-tRNA synthetase (PheRS), inter alia via its anticodon “GAA”, and then charged by PheRS with phenylalanine to form the corresponding aminoacyl-tRNA (e.g., phenylalanyl-tRNA). The tRNA_{GAA} aminoacylated or charged with Phe then recognizes the codon UUC (UUU) on the mRNA via its anticodon GAA (AAA) and phenylalanine can be introduced via peptide bonding into the growing polypeptide chain during the translation process of polypeptide synthesis. Likewise, the tRNA_{CUA} is the tRNA for the corresponding non-canonical amino acid pyrrolysine (Pyl), encoded by UAG. Pairs of aminoacyl-tRNA synthetases (aaRS) and its cognate tRNA which are charged by the aaRS with the corresponding amino acid are also referred to herein as “orthogonal pair”. In one embodiment of the present invention, when being expressed in a host cell as described and provided herein, the aminoacyl-tRNA synthetase described and provided herein is expressed together with its corresponding cognate tRNA as orthogonal pair.

In one embodiment of the present invention, the tRNA corresponding to an aliphatic amino acid which is aminoacylated by the aaRS encoded by a polynucleotide as described and provided herein may be a tRNA^{Pyl} (tRNA_{CUA}) (may also be referred to as tRNA^{Pyl}_{CUA}), and the corresponding aliphatic amino acid may be Pyl, and the aaRS may be a variant of pyrrolysyl tRNA synthetase (PyIRS). In this context of the present invention, the orthogonal pair would be PyIRS variant and its cognate tRNA^{Pyl} (tRNA_{CUA}). Accordingly, in one embodiment of the present invention, when being expressed in a host cell as described and provided herein, the PyIRS variant as described and provided herein is expressed in said host cell together with tRNA^{Pyl} (tRNA_{CUA}) as orthogonal pair. In a specific embodiment of the present invention, the PyIRS variant and/or the corresponding orthogonal tRNA^{Pyl} (tRNA_{CUA}) may be derived from Methanomethylophilus alvus Mx1201Ca.

The term “codon-optimized”, “codon-optimization”, etc. as used herein is generally known in the art and inter alia relates to the fact that different organisms use different codons for the same amino acid in different frequencies (“codon usage” as known in the art). Due to different codon usage, it is possible that a gene from one organism may hardly or differently be expressed in another organism as the other organism uses one or more codons more often, more rarely or not at all and, thus, may have only few or no corresponding tRNAs for such codons and, consequently, suitable aminoacyl-tRNA synthetases for such tRNAs. Accordingly, it may be required or at least advantageous to optimize the codons of a certain gene from one organism before transferring it to another organism in order to ensure comparable expression patterns. Such optimization usually takes place by substituting one or more bases within a nucleic acid sequence such as not to change its coded amino acid (i.e. silent mutation), but just to conform with the codon usage for a given amino acid codon with regard to the organism in which the nucleic acid molecule shall be expressed.

As such, as used herein, a given polynucleotide sequence may be considered “codon-optimized” to a selected host cell if it contains one or more codons for a given amino acid which is used with the highest frequency for said amino acid in said host cell. In one embodiment of the present invention, a given polynucleotide sequence may be considered “codon-optimized” to a selected host cell if it contains one or more respective codons used with the highest usage frequency in said selected host cell for 1, 2, 3 or all respective amino acids encoded by the polynucleotide. Alternatively, a codon usage frequency in the source organism can be used to adjust/optimize/harmonize the translation rate of the ribosome in the target organism so that it remains the same/similar/compatible to that in the source organism (e.g., to allow for a desired/correct folding of the polypeptide chain to take place). In a specific embodiment of the present invention, a given polynucleotide sequence may be considered “codon-optimized” to a selected host cell if it contains exclusively the respective codons with the highest usage frequency in the selected host cell for 1, 2, 3 or all respective encoded amino acids, i.e. no codon with a lower usage frequency for 1, 2, 3 or all respective encoded amino acids. Such codons with the highest frequency usage may be naturally contained in said polynucleotide or be introduced via suitable genetic modification tools known in the art (e.g., random or preferably site-specific mutations; PCR, restriction enzyme-based mutagenesis, CRISPR/Cas, etc). Codon-optimization may also and additionally refer to exchange of different stop codons. For example, if a given host cell expresses certain suppressor molecules for certain stop codons, the stop codon (e.g., amber, ochre or opal) may be adapted accordingly. Such process as defined herein above is referred to herein as “codon-optimization”. For many though not all organisms, there are codon usage tables available which show the codon usage frequency for the respective host cell, i.e. which codons are used more often than others (and at which ratio). However, such codon usage tables are not available for all organisms (e.g., not for Candidatus Methanomethylophilus alvus Mx1201Ca).

It is also one advantage of the present system that it is suitable to avoid interference with stop codons of the host cell while incorporating (preferably non-canonical) amino acids into polypeptides. For example, the aminoacyl-tRNA synthetase variants described and provided herein (and encoded by the polynucleotide of the present invention) may be a PyIRS variant, which is able to aminoacylate its cognate tRNA^{Pyl} (tRNA_{CUA}) with a lysine derivative as described herein (e.g., pyrrolysine or boc-lysine). As already mentioned, the anticodon CUA recognizes the amber stop codon (UAG) on the mRNA and, thus, acts as suppressor for the amber codon. In this context, when applied to host cells exhibiting a generally low level of amber stop codons within their respective genome, it is possible to avoid interference of incorporation of amino acids bound to their cognate tRNA (e.g., tRNA^{Pyl}) recognizing the amber stop codon.

As used herein, “silent” mutations mean base substitutions within a nucleic acid sequence which do not change the amino acid sequence encoded by the nucleic acid sequence. “Conservative” substitutions mean substitutions as listed as “Exemplary Substitutions” in Table I below. “Highly conservative” substitutions as used herein mean substitutions as shown under the heading “Preferred Substitutions” in Table I below.

The term “position” when used in accordance with the present invention may refer to a position of an amino acid within an amino acid sequence depicted herein. The term “corresponding” in this context may include that a position is not only determined by the number of the preceding nucleotides/amino acids.

TABLE I
Amino Acid Substitutions

| | Exemplary | Preferred |
|---|---|---|
| Original | Substitutions | Substitutions |
| Ala (A) | val; leu; ile | Val |
| Arg (R) | lys; gln; asn | lys |
| Asn (N) | gln; his; asp, lys; arg | gln |
| Asp (D) | glu; asn | glu |
| Cys (C) | ser, ala | ser |
| Gln (Q) | asn; glu | asn |
| Glu (E) | asp; gln | asp |
| Gly (G) | ala | ala |
| His (E) | asn; gln; lys; arg | arg |
| Ile (I) | leu; val; met; ala; phe; | leu |
| Leu (L) | norleucine; ile; val; met; ala; | ile |
| Lys (K) | arg; gin; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | Phe |
| Val (V) | ile; leu; met; phe; ala; | leu |

Moreover, the term “identity” as used herein may mean that there is a functional and/or structural equivalence between the corresponding sequences. Nucleic acid/amino acid sequences having the given identity levels to the herein-described particular nucleic acid/amino acid sequences may represent derivatives/variants of these sequences which, preferably, have the same biological function. They may be either naturally occurring variations, for instance sequences from other varieties, species, etc., or mutations, and said mutations may have formed naturally or may have been produced by deliberate mutagenesis. Furthermore, the variations may be synthetically produced sequences. The variants may be naturally occurring variants or synthetically produced variants or variants produced by recombinant DNA techniques. Deviations from the above-described nucleic acid sequences may have been produced, e.g., by deletion, substitution, addition, insertion and/or recombination. The term “addition” refers to adding at least one nucleic acid residue/amino acid to the end of the given sequence, whereas “insertion” refers to inserting at least one nucleic acid residue/amino acid within a given sequence. The term “deletion” refers to deleting or removal of at least one nucleic acid residue or amino acid residue in a given sequence. The term “substitution” refers to the replacement of at least one nucleic acid residue/amino acid residue in a given sequence. Again, these definitions as used here apply, mutatis mutandis, for all sequences provided and described herein.

Generally, as used herein, the terms “polynucleotide” and “nucleic acid” or “nucleic acid molecule” are to be construed synonymously. Generally, nucleic acid molecules may comprise inter alia DNA molecules, RNA molecules, oligonucleotide thiophosphates, substituted ribo-oligonucleotides or PNA molecules. Furthermore, the term “nucleic acid molecule” may refer to DNA or RNA or hybrids thereof or any modification thereof that is known in the art (see, e.g., U.S. Pat. Nos. 5,525,711, 4,711,955, 5,792,608 or EP 302175 for examples of modifications). The polynucleotide sequence may be single- or double-stranded, linear or circular, natural or synthetic, and without any size limitation. For instance, the polynucleotide sequence may be genomic DNA, cDNA, mitochondrial DNA, mRNA, antisense RNA, ribozymal RNA or a DNA encoding such RNAs or chimeroplasts (Gamper et al., Nucleic Acids Res (2000), 28 (21): 4332-4339). Said polynucleotide sequence may be in the form of a vector, plasmid or of viral DNA or RNA. Also described herein are nucleic acid molecules which are complementary to the nucleic acid molecules described above and nucleic acid molecules which are able to hybridize to nucleic acid molecules described herein. A nucleic acid molecule described herein may also be a fragment of the nucleic acid molecules in context of the present invention. Particularly, such a fragment is a functional fragment. Examples for such functional fragments are nucleic acid molecules which can serve as primers.

The term “hybridization” or “hybridizes” as used herein in context of nucleic acid molecules/DNA sequences may relate to hybridizations under stringent or non-stringent conditions. If not further specified, the conditions are preferably non-stringent. Said hybridization conditions may be established according to conventional protocols described, for example, in Sambrook, Russell “Molecular Cloning, A Laboratory Manual”, Cold Spring Harbor Laboratory, N. Y. (2001); Current Protocols in Molecular Biology, Update May 9, 2012, Print ISSN: 1934-3639, Online ISSN: 1934-3647; Ausubel, “Current Protocols in Molecular Biology”, Green Publishing Associates and Wiley Interscience, N. Y. (1989), or Higgins and Hames (Eds.) “Nucleic acid hybridization, a practical approach” IRL Press Oxford, Washington D.C., (1985). The setting of conditions is well within the skill of the artisan and can be determined according to protocols described in the art. Thus, the detection of only specifically hybridizing sequences will usually require stringent hybridization and washing conditions such as 0.1×SSC, 0.1% SDS at 65° C. Non-stringent hybridization conditions for the detection of homologous or not exactly complementary sequences may be set at 6×SSC, 1% SDS at 65° C. As is well known, the length of the probe and the composition of the nucleic acid to be determined constitute further parameters of the hybridization conditions. Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility. In accordance to the invention described herein, low stringent hybridization conditions for the detection of homologous or not exactly complementary sequences may, for example, be set at 6×SSC, 1% SDS at 65° C. As is well known, the length of the probe and the composition of the nucleic acid to be determined constitute further parameters of the hybridization conditions.

Hybridizing nucleic acid molecules also comprise fragments of the above described molecules. Such fragments may represent nucleic acid molecules which code for a functional aaRS as described herein or a functional fragment thereof which can serve as a primer. Furthermore, nucleic acid molecules which hybridize with any of the aforementioned nucleic acid molecules also include complementary fragments, derivatives and variants of these molecules. Additionally, a hybridization complex refers to a complex between two nucleic acid sequences by virtue of the formation of hydrogen bonds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., Cot or Rot analysis) or between one nucleic acid sequence present in solution and another nucleic acid sequence immobilized on a solid support (e.g., membranes, filters, chips, pins or glass slides to which, e.g., cells have been fixed). The terms complementary or complementarity refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between single-stranded molecules. The degree of complementarity between nucleic acid strands has significant effects on the efficiency and strength of hybridization between nucleic acid strands. This is of particular importance in amplification reactions, which depend upon binding between nucleic acids strands. The term “hybridizing sequences” preferably refers to sequences which display a sequence identity of at least 45%, more preferably at least 50%, more preferably at least 55%, more preferably at least 60%, more preferably at least 65%, more preferably at least 70%, more preferably at least 75%, more preferably at least 80%. more preferably at least 85%, more preferably at least 90%, more preferably at least 95%, more preferably at least 96%, more preferably at least 97%, more preferably at least 98% more preferably at least 99%, more preferably at least 99.5%, and most preferably 100% identity with a nucleic acid sequence as described herein encoding an aaRS as described and provided herein.

In one embodiment of the present invention, the amino acid which is aminoacylated with its corresponding tRNA by the aaRS variant as described and provided herein may be a lysine or, preferably, a lysine derivative. In one embodiment of the present invention, such lysine derivative may be selected from the group consisting of pyrrolysine (Pyl), boc-lysine (Nₐ-(tert-Butoxycarbonyl)-L-lysine), alloc-lysine (N₆—Alloc-L-lysine), azide-lysine, 2-N,6-N-Bis(2,3-dihydroxy-N-benzoyl)-L-serine, 2-N,6-N-Bis(2,3-dihydroxy-N-benzoyl)-L-serine amide, 3-hydroxylysine, N-benzoylglycyl-N⁶-[2-hydroxy-2-(3-methylquinoxalin-2-yl)ethyl]lysine, N-benzoylglycyl-N⁶-[2-hydroxy-3-(quinoxalin-2-yl)propyl]lysine, N-hippuryl-N⁶-(carboxymethyl)lysine, N⁶-(2,4-dinitrophenyl)lysine, N⁶-(2-carboxyethyl)lysine, N⁶-acetonyllysine, N⁶-carbamoylmethyllysine, N⁶-methyllysine, hydroxylysine, isodesmosine, ornithine derivatives such as 2-amino-5-(prop-2-ynoylamino)pentanoic acid (5-(prop-2-ynoylamino)ornithine), and 2-amino-5-[(azidoacetyl)amino]pentanoic acid. In a specific embodiment, the lysine derivative is pyrrolysine or boc-lysine.

The present invention also relates to a vector comprising a polynucleotide described and provided in accordance with the present invention. That is, the present invention also relates to a vector comprising a polynucleotide as described and provided herein encoding an aminoacyl-tRNA synthetase variant (aaRS) capable of catalyzing the aminoacylation of its cognate tRNA with an aliphatic or aromatic amino acid to form an aminoacyl-tRNA as described and also provided herein, said polynucleotide comprising a nucleotide sequence, wherein said nucleotide sequence is codon-optimized to a selected host cell which is not Candidatus Methanomethylophilus alvus Mx1201Ca.

The present invention also relates to a vector comprising a polynucleotide encoding a tRNA which corresponds to the amino acid with which said tRNA is aminoacylated by the aminoacyl-tRNA synthetase (aaRS) variant as described and provided herein.

The vector may comprise such polynucleotide encoding a tRNA as described above in addition to the polynucleotide encoding the aaRS variant as described further above, wherein both polynucleotides may be under the control of the same or different promoters. In context with the present invention, such tRNAs may also comprise mutated or engineered tRNAs which exhibit altered (e.g., higher) binding affinity or substrate specificity to its corresponding aaRS or altered (e.g., higher) to its corresponding amino acid, or altered anticodon-codon binding behaviors. Such altered anticodon-codon binding behaviors may be in such a way that specific codons are recognized more specifically by the tRNA's anticodon (i.e. higher base-to-base binding specificity, less wobble-base pairing abilities), e.g., either by altering the anticodon itself or by adapting the special structure of the tRNA such as to alter the binding behavior. Methods for engineering tRNAs in this context are known in the art and comprise those as described in, e.g., Liu et al., PNAS (1997), 94: 10092-10097; Wang and Schultz, Chem Biol (2001), 8: 883-890; and Maranhao et al., ACS Synth Biol (2017), 6 (1): 108-119. Also, in context with the present invention, the tRNAs may be engineered such as to enhance the affinity of aminoacylated tRNA to the EF (elongation factor) Tu (cf., e.g., Schrader et al., PNAS (2011), 108 (13): 5215-5220).

The term “vector” as used herein particularly refers to plasmids, cosmids, viruses, bacteriophages and other vectors commonly used in genetic engineering. In one embodiment of the present invention, the vectors are suitable for the transformation, transduction and/or transfection of host cells as described herein, e.g., prokaryotic cells (e.g., (eu)bacteria, archaea), eukaryotic cells (e.g., mammalian cells, insect cells) fungal cells, yeast, and the like. Examples of bacterial host cells in context with the present invention comprise Gram negative and Gram positive cells. Specific examples for suitable host cells may comprise inter alia E. coli, Mycoplasma capricolum, SF9 cells, CHO, C. elegans cell, S. cerevisiae, Schizosaccharmyces pombe, Micrococcus luteus, Corynebacterium glutamicum, Pichia pastoris (today also known as Komagataella pastoris or Komagataella phaffii), plant cells, and Bombyx mori. In one embodiment of the present invention, said vectors are suitable for stable transformation of the host cells, for example to express the aaRS and/or the tRNA as described and provided herein.

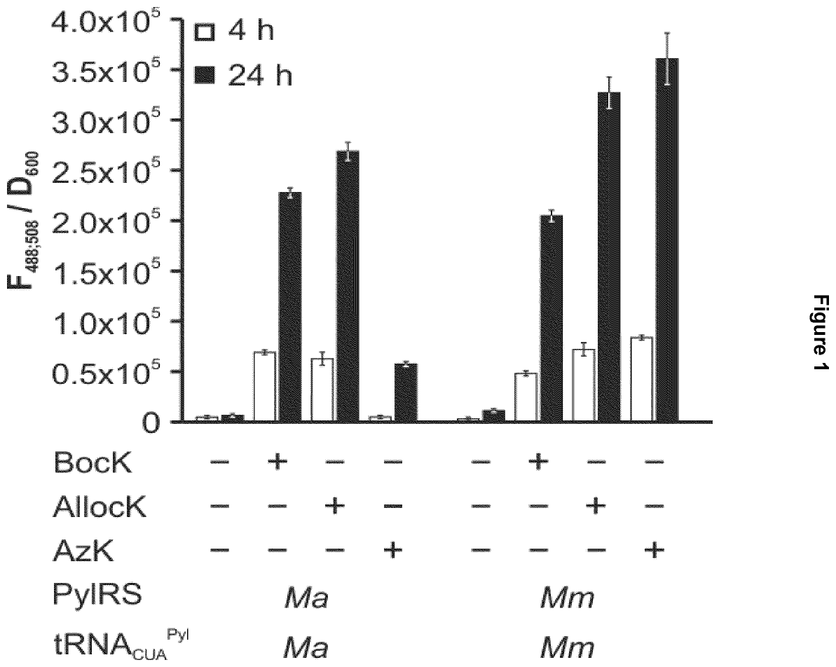

Accordingly, in one aspect of the invention, the vector as provided is an expression vector. Generally, expression vectors have been widely described in the literature. As a rule, they may not only contain a selection marker gene and a replication-origin ensuring replication in the host selected, but also a promoter, and in most cases a termination signal for transcription. Between the promoter and the termination signal there is preferably at least one restriction site or a polylinker which enables the insertion of a nucleic acid sequence/molecule desired to be expressed. It is to be understood that a vector can also be generated by taking advantage of an expression vector known in the prior art that already comprises a promoter suitable to be employed in context of this invention, for example, for expression of an aaRS and/or tRNA as described herein. The nucleic acid construct is preferably inserted into that vector in a manner the resulting vector comprises only one promoter suitable to be employed in context of this invention. The skilled person knows how such insertion can be put into practice. For example, the promoter can be excised either from the nucleic acid construct or from the expression vector prior to ligation. In one embodiment of the present invention, the vector is able to integrate into the host cell genome. The vector may be any vector suitable for the respective host cell, preferably an expression vector. The vector may comprise the polynucleotide encoding an aminoacyl-tRNA synthetase (aaRS) capable of catalyzing the aminoacylation of its cognate tRNA with an aliphatic or aromatic amino acid to form an aminoacyl-tRNA as described and also provided herein, and/or the cognate tRNA as described and provided herein. A non-limiting example of the vector of the present invention may comprise pSCS (see, e.g., FIG. 1) comprising the polynucleotide in context of the present invention.

In one embodiment of the present invention, the host cell comprises a polynucleotide encoding the aaRS variant as described and provided herein, and the corresponding tRNA as also described herein, for example as orthogonal pair (e.g., MmaPyIRS with MmatRNA^{Pyl}) or as hybrid pair (e.g., MmaPyIRS with a tRNA^{Pyl} derived from another organism than Mma, such as tRNA^{Pyl} derived from Mm, or vice versa).

The host cell of the present invention may generally be any host cell, preferably being capable of stably expressing a polynucleotide encoding an aaRS and/or a tRNA as described and provided herein. Such host cells may comprise, inter alia, e.g., prokaryotic cells (e.g., (eu)bacteria, archaea), eukaryotic cells (e.g., mammalian cells, insect cells) fungal cells, yeast, or entire organisms (e.g., Bombyx mori) and the like. In one embodiment of the present invention, the host cell is not archaea. Examples of bacterial host cells in context with the present invention comprise Gram negative and Gram positive cells. Specific examples for suitable host cells may comprise inter alia E. coli, Corynebacterium glutamicum, Mycoplasma capricolum, CHO, SF9 cells, C. elegans cell, S. cerevisiae, Schizosaccharmyces pombe, Micrococcus luteus, Pichia pastoris (today also known as Komagataella pastoris or Komagataella phaffii), plant cells, and Bombyx mori.

The present invention further relates to a polypeptide encoded by a polynucleotide as described and provided herein, particularly a polynucleotide encoding an aaRS variant and/or a tRNA as described and provided herein.

The present invention also relates to a composition comprising an orthogonal pair of an aminoacyl-tRNA synthetase variant as described and provided herein and its cognate tRNA as described and provided herein.

The present invention further relates to the use of a polynucleotide encoding an aminoacyl-tRNA synthetase (aaRS) variant as described and provided herein, the use of a vector comprising such polynucleotide, the use of a host cell comprising such vector and/or polynucleotide, the use of a polypeptide encoded by such polynucleotide or an aminoacyl-tRNA synthetase as described and provided herein, to catalyse the introduction of an aliphatic or aromatic amino acid as described herein into a polypeptide. As known in the art, such introduction usually takes place during the translation process of polypeptide biosynthesis.

The present invention further relates to the use of a polynucleotide encoding a tRNA as described and provided herein, the use of a vector comprising such polynucleotide, the use of a host cell comprising such vector and/or polynucleotide, the use of a polypeptide encoded by such polynucleotide or an aminoacyl-tRNA synthetase as described and provided herein, to catalyse the introduction of an aliphatic or aromatic amino acid as described herein into a polypeptide. As known in the art, such introduction usually takes place during the translation process of polypeptide biosynthesis.

The present invention further relates to the use of an orthogonal pair of aminoacyl-tRNA synthetase variant and its cognate tRNA as described and provided herein to catalyse the introduction of an aliphatic or aromatic amino acid as described herein into a polypeptide. As known in the art, such introduction usually takes place during the translation process of polypeptide biosynthesis.

The present invention also relates to a method of incorporating an aliphatic or aromatic amino acid as described herein into a polypeptide, comprising the following steps:
(i) expressing a polynucleotide encoding an aminoacyl-tRNA synthetase variant as described and provided herein, and
(ii) expressing a polynucleotide encoding a tRNA (which is cognate to the aminoacyl-tRNA synthetase variant as described and provided herein), said aminoacyl-tRNA synthetase variant and tRNA being an orthogonal pair.

The embodiments which characterize the present invention are described herein, shown in the Figures, illustrated in the Examples, and reflected in the claims.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

Methods for preparing aminoacyl-tRNA synthetase variants of the invention (e.g., as described in WO02092797). Several methods for introducing mutations into genes are known in the art. After a brief description of cloning of aminoacyl-tRNA synthetase-encoding DNA sequences, methods for generating mutations at specific sites within the aminoacyl-tRNA synthetase-encoding sequence will be described.

Cloning a DNA sequence encoding an aminoacyl-tRNA synthetase The DNA sequence encoding a parent aminoacyl-tRNA synthetase may be isolated from any cell or microorganism producing the aminoacyl-tRNA synthetase in question, using various methods well known in the art. First, a genomic DNA and/or cDNA library should be constructed using chromosomal DNA or messenger RNA from the organism that produces the aminoacyl-tRNA synthetase to be studied.

Then, if the amino acid sequence of the aminoacyl-tRNA synthetase is known, homologous, labeled oligonucleotide probes may be synthesized and used to identify aminoacyl-tRNA synthetase-encoding clones from a genomic library prepared from the organism in question. Alternatively, a labeled oligonucleotide probe containing sequences homologous to a known aminoacyl-tRNA synthetase gene could be used as a probe to identify aminoacyl-tRNA synthetase-encoding clones, using hybridization and washing conditions of lower stringency.

Yet another method for identifying aminoacyl-tRNA synthetase-encoding clones would involve inserting fragments of genomic DNA into an expression vector, such as a plasmid, transforming aminoacyl-tRNA synthetase-negative bacteria with the resulting genomic DNA library, and then plating the transformed bacteria onto agar containing a substrate for aminoacyl-tRNA synthetase, thereby allowing clones expressing the aminoacyl-tRNA synthetase to be identified. Alternatively, the DNA sequence encoding the enzyme may be prepared synthetically by established standard methods, e.g., the phosphoroamidite method described by Beaucage et al., Tetrahedron Lett (1981), 22 (20): 1859-1862, or the method described by Matthes et al., EMBO J (1984), 3 (4): 801-805. In the phosphoramidite method, oligonucleotides are synthesized, e.g., in an automatic DNA synthesizer, purified, annealed, ligated and cloned in appropriate vectors. Finally, the DNA sequence may be of mixed genomic and synthetic origin, mixed synthetic and cDNA origin or mixed genomic and cDNA origin, prepared by ligating fragments of synthetic, genomic or cDNA origin (as appropriate, the fragments corresponding to various parts of the entire DNA sequence), in accordance with standard techniques. The DNA sequence may also be prepared by polymerase chain reaction (PCR) using specific primers, for instance as described in U.S. Pat. No. 4,683,202 or Saiki et al., Science (1988), 239 (4839): 487-491.

Site-directed mutagenesis: Once an aminoacyl-tRNA synthetase-encoding DNA sequence has been isolated, and desirable sites for mutation identified, mutations may be introduced using synthetic oligonucleotides. These oligonucleotides contain nucleotide sequences flanking the desired mutation sites; mutant nucleotides are inserted during oligonucleotide synthesis. In a specific method, a single stranded gap of DNA, bridging the aminoacyl-tRNA synthetase-encoding sequence, is created in a vector carrying the aminoacyl-tRNA synthetase gene. Then the synthetic nucleotide, bearing the desired mutation, is annealed to a homologous portion of the single-stranded DNA. The remaining gap is then filled in with DNA polymerase I (Klenow fragment) and the construct is ligated using T4 ligase. A specific example of this method is described in Morinaga et al., Biotechnology (N Y) (1984), 2 (7): 636-639. U.S. Pat. No. 4,760,025 disclose the introduction of oligonucleotides encoding multiple mutations by performing minor alterations of the cassette. However, an even greater variety of mutations can be introduced at any one time by the Morinaga method, because a multitude of oligonucleotides, of various lengths, can be introduced.

Another method for introducing mutations into aminoacyl-tRNA synthetase-encoding DNA sequences is described in Nelson et al., Anal Biochem (1989), 180 (1): 147-151. It involves the 3-step generation of a PCR fragment containing the desired mutation introduced by using a chemically synthesized DNA strand as one of the primers in the PCR reactions. From the PCR-generated fragment, a DNA fragment carrying the mutation may be isolated by cleavage with restriction endonucleases and reinserted into an expression plasmid.

Alternative methods for providing variants of the invention include gene shuffling, e.g., as described in WO 95/22625 (from Affymax Technologies N. V.) or in WO9707205 (from Novo Nordisk A/S), mutant gene synthesis, or other corresponding techniques resulting in a hybrid enzyme comprising the mutation (s), e.g., substitution (s) and/or deletion (s), in question.

Expression of aminoacyl-tRNA synthetase variants: According to the invention, a DNA sequence encoding the variant produced by methods described above, or by any alternative methods known in the art, can be expressed, in enzyme form, using an expression vector which typically includes control sequences encoding a promoter, operator, ribosome binding site, translation initiation signal, and, optionally, a repressor gene or various activator genes.

The recombinant expression vector carrying the DNA sequence encoding an aminoacyl-tRNA synthetase variant of the invention may be any vector, which may conveniently be subjected to recombinant DNA procedures, and the choice of vector will often depend on the host cell into which it is to be introduced. Thus, the vector may be an autonomously replicating vector, i.e., a vector which exists as an extrachromosomal entity, the replication of which is independent of chromosomal replication, e.g., a plasmid, a bacteriophage or an extrachromosomal element, minichromosome or an artificial chromosome. Alternatively, the vector may be one which, when introduced into a host cell, is integrated into the host cell genome and replicated together with the chromosome(s) into which it has been integrated.

In the vector, the DNA sequence should be operably connected to a suitable promoter sequence. The promoter may be any DNA sequence, which shows transcriptional activity in the host cell of choice and may be derived from genes encoding proteins either homologous or heterologous to the host cell, or may be entirely synthetic.

The expression vector of the invention may also comprise a suitable transcription terminator and, in eukaryotes, polyadenylation sequences operably connected to the DNA sequence encoding the aminoacyl-tRNA synthetase variant of the invention. Termination and polyadenylation sequences may suitably be derived from the same sources as the promoter.

The vector may further comprise a DNA sequence enabling the vector to replicate in the host cell in question. Examples of such sequences are the origins of replication of plasmids pUC19, pBR322, pET vector series, pACYC177, pUB110, pE194, pAMB1 and plJ702.

The vector may also comprise a selectable marker, e.g. a gene the product of which complements a defect in the host cell or furnishes the cells with a (growth) advantage when maintained.

The procedures used to ligate the DNA construct of the invention encoding an aminoacyl-tRNA synthetase variant, the promoter, terminator and other elements, respectively, and to insert them into suitable vectors containing the information necessary for replication, are well known to persons skilled in the art (cf., for instance, Sambrook et al., Molecular Cloning: A Laboratory Manual, 4th Ed., Cold Spring Harbor, 2012).

In some aspects the present invention relates to/provides a variant of a parent aminoacyl-tRNA synthetase (e.g., EC:6.1.1.-.), wherein said variant comprises: a substitution at one or more positions corresponding to positions 168 and 129 of the amino acid sequence set forth in SEQ ID NO: 1 or UniProtKB Accession Number: M9SC49 (e.g., MaPyIRS), (e.g., using the numbering of SEQ ID NO: 1 or UniProtKB Accession Number: M9SC49); preferably wherein said variant comprises the following combination of substitutions: X168C+X129L (e.g., V168C+M129L), wherein said variant is a polypeptide having at least 70%, e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, but less than 100% sequence identity with the amino acid sequence set forth in SEQ ID NO: 1 or UniProtKB Accession Number: M9SC49 (e.g., MaPyIRS); further preferably wherein said variant has aminoacyl-tRNA synthetase activity (e.g., EC:6.1.1.-.).

In some aspects the present invention relates to/provides a method for obtaining an aminoacyl-tRNA synthetase variant (e.g., EC:6.1.1.-.), said method comprising: (i) introducing into a parent aminoacyl-tRNA synthetase a substitution at one or more positions corresponding to positions 168 and 129 of the polypeptide of SEQ ID NO: 1 or UniProtKB Accession Number: M9SC49 (e.g., MaPyIRS), (e.g., using the numbering of SEQ ID NO: 1 or UniProtKB Accession Number: M9SC49); preferably wherein said variant has aminoacyl-tRNA synthetase activity (e.g., EC:6.1.1.-.), further preferably wherein said variant comprises the following combination of substitutions: X168C+X129L (e.g., V168C+M129L); and (ii) recovering said variant.

In some aspects the present invention relates to/provides a variant or method of the present invention, wherein said variant comprises the following combination of substitutions: V168C+M129L, preferably corresponding to positions of the amino acid sequence as set forth in SEQ ID NO: 1 or UniProtKB Accession Number: M9SC49 (e.g., using the numbering of SEQ ID NO: 1 or UniProtKB Accession Number: M9SC49).

In some aspects the present invention relates to/provides a variant or method of the present invention, wherein said variant is a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, or 100% sequence identity with the amino acid sequence as set forth in SEQ ID NO: 2 (e.g., MaPyIRS having V168C+M129L substitutions).

In some aspects the present invention relates to/provides a variant or method of the present invention, wherein said variant comprises or consists of SEQ ID NO: 2.

In some aspects the present invention relates to/provides a variant or method of the present invention, wherein said variant is capable of catalyzing the aminoacylation of its cognate tRNA with an aromatic and/or aliphatic amino acid, e.g., to form an aminoacyl-tRNA.

In some aspects the present invention relates to/provides a variant or method of the present invention, wherein said amino acid is a lysine derivative, preferably said variant comprises a combination of substitutions X168C+X129L (e.g., V168C and M129L).

In some aspects the present invention relates to/provides a variant or method of the present invention, wherein said lysine derivative is selected from the group consisting of: N^{ε}-((2-azidoethoxy)carbonyl)-L-lysine (AzK), pyrrolysine, boc-lysine, alloc-lysine, azide-lysine, 2-N,6-N-Bis(2,3-dihydroxy-N-benzoyl)-L-serine, 2-N,6-N-Bis(2,3-dihydroxy-N-benzoyl)-L-serine amide, 3-hydroxylysine, N-benzoylglycyl-N⁶-[2-hydroxy-2-(3-methylquinoxalin-2-yl)ethyl]lysine, N-benzoylglycyl-N⁶-[2-hydroxy-3-(quinoxalin-2-yl)propyl]lysine, N-hippuryl-N⁶-(carboxymethyl)lysine, N⁶-(2,4-dinitrophenyl)lysine, N⁶-(2-carboxyethyl)lysine, N⁶-acetonyllysine, N⁶-carbamoylmethyllysine, N⁶-methyllysine, hydroxylysine, isodesmosine, ornithine derivatives such as 2-amino-5-(prop-2-ynoylamino)pentanoic acid (5-(prop-2-ynoylamino)ornithine), and 2-amino-5-[(azidoacetyl)amino]pentanoic acid.

In some aspects the present invention relates to/provides a variant or method of the present invention, wherein said lysine derivative is N^{ε}-((2-azidoethoxy)carbonyl)-L-lysine (e.g., AzK), preferably said variant comprises a combination of substitutions X168C+X129L (e.g., V168C and M129L).

In some aspects the present invention relates to/provides a variant or method of the present invention, wherein said variant is capable of accepting non-canonical substrates, preferably non-canonical amino acids (ncAAs) (e.g., ncAAs with reactive bioorthogonal groups, e.g., in the side chain, e.g., an azido-group).

In some aspects the present invention relates to/provides a variant or method of the present invention, wherein said parent aminoacyl-tRNA synthetase (e.g., EC:6.1.1.-.) is obtainable from a Methanomethylophilus sp. (e.g., Methanomethylophilus alvus, e.g., Methanomethylophilus alvus Mx1201Ca).

In some aspects the present invention relates to/provides a variant or method of the present invention, wherein said parent aminoacyl-tRNA synthetase (e.g., EC:6.1.1.-.) is a polypeptide having at least 70%, e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, but less than 100% sequence identity with the amino acid sequence set forth in SEQ ID NO: 1 or UniProtKB Accession Number: M9SC49 (e.g., MaPyIRS); or pyrrolysine—tRNA ligase from: Methanosarcina barkeri (e.g., UniProtKB—Q6WRH6); Methanosarcina barkeri (strain Fusaro/DSM 804) (e.g., UniProtKB—Q46E77); Methanosarcina acetivorans (strain ATCC 35395/DSM 2834/JCM 12185/C2A) (e.g., UniProtKB—Q8TUB8); Methanosarcina mazei (strain ATCC BAA-159/DSM 3647/Goe1/Go1/JCM 11833/OCM 88) (Methanosarcina frisia) (e.g., UniProtKB—Q8PWY1); Methanococcoides burtonii (strain DSM 6242/NBRC 107633/OCM 468/ACE-M) (e.g., UniProtKB—Q12UB6); or Methanosarcina thermophila (e.g., UniProtKB—Q1 L6A3).

In some aspects the present invention relates to/provides a variant or method of the present invention, wherein said parent aminoacyl-tRNA synthetase (e.g., EC:6.1.1.-.) has the amino acid sequence set forth in SEQ ID NO: 1 or UniProtKB Accession Number: M9SC49 (e.g., MaPyIRS) or pyrrolysine—tRNA ligase from: Methanosarcina barkeri (e.g., UniProtKB—Q6WRH6); Methanosarcina barkeri (strain Fusaro/DSM 804) (e.g., UniProtKB—Q46E77), Methanosarcina acetivorans (strain ATCC 35395/DSM 2834/JCM 12185/C2A) (e.g., UniProtKB—Q8TUB8); Methanosarcina mazei (strain ATCC BAA-159/DSM 3647/Goe1/Go1/JCM 11833/OCM 88) (Methanosarcina frisia) (e.g., UniProtKB—Q8PWY1); Methanococcoides burtonii (strain DSM 6242/NBRC 107633/OCM 468/ACE-M) (e.g., UniProtKB—Q12UB6); or Methanosarcina thermophila (e.g., UniProtKB—Q1 L6A3).

In some aspects the present invention relates to/provides a variant or method of the present invention, wherein said aminoacyl-tRNA synthetase activity (e.g., EC:6.1.1.-.) comprises or consists of a pyrrolysyl-tRNA synthetase activity (e.g., EC:6.1.1.26).

In some aspects the present invention relates to/provides a variant or method of the present invention, wherein said aminoacyl-tRNA synthetase variant (e.g., EC:6.1.1.-.) is a pyrrolysyl-tRNA synthetase variant (e.g., EC:6.1.1.26), preferably having SEQ ID NO: 3.

In some aspects the present invention relates to/provides polynucleotide encoding the variant of the present invention (e.g., as disclosed WO 2018/185222 A1).

In some aspects the present invention relates to/provides a nucleic acid construct comprising the polynucleotide of the present invention.

In some aspects the present invention relates to/provides an expression vector comprising the polynucleotide and/nucleic acid construct of the present invention.

In some aspects the present invention relates to/provides a host cell comprising at least one of the following: i) the variant of the present invention; ii) the polynucleotide of the present invention; iii) the nucleic acid construct of the present invention; and/or iv) the expression vector of the present invention.

In some aspects the present invention relates to/provides a host cell of the present invention, wherein said host cell is a recombinant (e.g., genetically modified, e.g., comprising a recombinant nucleic acid, e.g., a nucleic acid not derived from said host cell) host cell, preferably an isolated recombinant host cell.

In some aspects the present invention relates to/provides a host cell of the present invention, wherein said host cell is selected from the group consisting of: Methanomethylophilus sp. (e.g., Methanomethylophilus alvus, e.g., Methanomethylophilus alvus Mx1201Ca), E. coli, Corynebacterium glutamicum, Mycoplasma capricolum, CHO, SF9 cells, C. elegans cell, S. cerevisiae, Schizosaccharmyces pombe, Micrococcus luteus, Komagataella pastoris (e.g., Komagataella phaffii), and Bombyx mori.

In some aspects the present invention relates to/provides a method for producing the variant of the present invention, comprising: (i) cultivating the host cell of the present invention under conditions suitable for expression of said variant; and (ii) recovering said variant.

In some aspects the present invention relates to/provides a composition comprising at least one of the following: (i) variant of the present invention, preferably with a tRNA as orthogonal pair; (ii) a polynucleotide of the present invention; (iii) a nucleic acid construct of the present invention; (iv) an expression vector of the present invention; and/or (v) a host cell of the present invention.

In some aspects the present invention relates to/provides use of the variant, polynucleotide, nucleic acid construct, expression vector, host cell or composition of the present invention, for one or more of the following: (i) catalysing the aminoacylation of its cognate tRNA with an aromatic and/or aliphatic amino acid, e.g., to form an aminoacyl-tRNA; (ii) catalysing the introduction of an aromatic and/or aliphatic amino acid, e.g., into a polypeptide; (iii) site-specific incorporation of non-canonical substrates (e.g., non-canonical amino acids (ncAAs), e.g., with reactive bioorthogonal groups, e.g., in the side chain, such as an azido-group), e.g., into polypeptides, e.g., in response to an amber stop codon.

In some aspects the present invention relates to/provides a method for incorporating an aromatic and/or aliphatic amino acid, e.g., into a polypeptide, comprising the following steps: (i) expressing the polynucleotide, nucleic acid construct and/or vector of the present invention; or providing the variant of the present invention; and (ii) expressing a polynucleotide encoding a tRNA and/or providing said tRNA, said tRNA and the aminoacyl-tRNA synthetase variant provided in (i) or encoded by the polynucleotide, nucleic acid construct or vector in (i) being an orthogonal pair.

In some aspects the present invention relates to/provides a method, system or kit for incorporating an aromatic or aliphatic amino acid, e.g., into a polypeptide, comprising: (i) a variant, polynucleotide, nucleic acid construct, expression vector, host cell and/or composition of the present invention; (ii) a corresponding amber suppressor tRNA (e.g., orthogonal pair, o-pair, OP).

In some aspects the present invention relates to/provides a use or method of the present invention, wherein said use or method is an in vitro, ex vivo or in vivo use or method.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The invention is also characterized by the following items:
1. A variant of a parent aminoacyl-tRNA synthetase (e.g., EC:6.1.1.-.), wherein said variant comprises: a substitution at one or more positions corresponding to positions 168 and 129 of the amino acid sequence set forth in SEQ ID NO: 1 or UniProtKB Accession Number: M9SC49 (e.g., MaPyIRS), (e.g., using the numbering of SEQ ID NO: 1 or UniProtKB Accession Number: M9SC49), wherein said variant comprises the following combination of substitutions: X168C+X129L (e.g., C in position 168 and L in position 129 e.g., V168C+M129L), wherein said variant is a polypeptide having at least 70%, e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, but less than 100% sequence identity with the amino acid sequence set forth in SEQ ID NO: 1 or UniProtKB Accession Number: M9SC49 (e.g., MaPyIRS), wherein said variant has aminoacyl-tRNA synthetase activity (e.g., EC:6.1.1.-.).
2. A method for obtaining an aminoacyl-tRNA synthetase variant (e.g., EC:6.1.1.-.), said method comprising:
   i) introducing into a parent aminoacyl-tRNA synthetase a substitution at one or more positions corresponding to positions 168 and 129 of the polypeptide of SEQ ID NO: 1 or UniProtKB Accession Number: M9SC49 (e.g., MaPyIRS), (e.g., using the numbering of SEQ ID NO: 1 or UniProtKB Accession Number: M9SC49), wherein said variant has aminoacyl-tRNA synthetase activity (e.g., EC:6.1.1.-.), wherein said variant comprises the following combination of substitutions: X168C+X129L (e.g., V168C+M129L)
   ii) recovering said variant.
3. The variant or method according any one of the preceding items, wherein said variant comprises the following combination of substitutions: V168C+M129L, corresponding to positions of the amino acid sequence as set forth in SEQ ID NO: 1 or UniProtKB Accession Number: M9SC49 (e.g., using the numbering of SEQ ID NO: 1 or UniProtKB Accession Number: M9SC49).
4. The variant or method according any one of the preceding items, wherein said variant is a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, or 100% sequence identity with the amino acid sequence as set forth in SEQ ID NO: 2 (e.g., MaPyIRS having V168C+M129L substitutions).
5. The variant or method according any one of the preceding items, wherein said variant comprises or consists of SEQ ID NO: 2.
6. The variant or method according any one of the preceding items, wherein said variant is capable of catalyzing the aminoacylation of its cognate tRNA with an aromatic or aliphatic amino acid to form an aminoacyl-tRNA.
7. The variant or method according any one of the preceding items, wherein said amino acid is a lysine derivative, preferably said variant comprises a combination of substitutions X168C+X129L (e.g., V168C and M129L).
8. The variant or method according any one of the preceding items, wherein said lysine derivative is selected from the group consisting of: N^{ε}-((2-azidoethoxy)carbonyl)-L-lysine (AzK), pyrrolysine, boc-lysine, alloc-lysine, azide-lysine, 2-N,6-N-Bis(2,3-dihydroxy-N-benzoyl)-L-serine, 2-N,6-N-Bis(2,3-dihydroxy-N-benzoyl)-L-serine amide, 3-hydroxylysine, N-benzoylglycyl-N⁶-[2-hydroxy-2-(3-methylquinoxalin-2-yl)ethyl]lysine, N-benzoylglycyl-N⁶-[2-hydroxy-3-(quinoxalin-2-yl)propyl]lysine, N-hippuryl-N⁶-(carboxymethyl)lysine, N⁶-(2,4-dinitrophenyl)lysine, N⁶-(2-carboxyethyl)lysine, N-acetonyllysine, N⁶-carbamoylmethyllysine, N⁶-methyllysine, hydroxylysine, isodesmosine, ornithine derivatives such as 2-amino-5-(prop-2-ynoylamino)pentanoic acid (5-(prop-2-ynoylamino)ornithine), and 2-amino-5-[(azidoacetyl)amino]pentanoic acid.
9. The variant or method according any one of the preceding items, wherein said lysine derivative is N^{ε}-((2-azidoethoxy)carbonyl)-L-lysine (e.g., AzK), preferably said variant comprises a combination of substitutions X168C+X129L (e.g., V168C and M129L).
10. The variant or method according any one of the preceding items, wherein said variant is capable of accepting non-canonical substrates, preferably non-canonical amino acids (ncAAs) (e.g., ncAAs with reactive bioorthogonal groups in the side chain, e.g., an azido-group).
11. The variant or method according any one of the preceding items, wherein said parent aminoacyl-tRNA synthetase (e.g., EC:6.1.1.-.) is obtainable from a Methanomethylophilus sp. (e.g., Methanomethylophilus alvus, e.g., Methanomethylophilus alvus Mx1201Ca).
12. The variant or method according any one of the preceding items, wherein said parent aminoacyl-tRNA synthetase (e.g., EC:6.1.1.-.) is a polypeptide having at least 70%, e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, but less than 100% sequence identity with the amino acid sequence set forth in SEQ ID NO: 1 or UniProtKB Accession Number: M9SC49 (e.g., MaPyIRS).
13. The variant or method according any one of the preceding items, wherein said parent aminoacyl-tRNA synthetase (e.g., EC:6.1.1.-.) has the amino acid sequence set forth in SEQ ID NO: 1 or UniProtKB Accession Number: M9SC49 (e.g., MaPyIRS).
14. The variant or method according any one of the preceding items, wherein said aminoacyl-tRNA synthetase activity (e.g., EC:6.1.1.-.) comprises a pyrrolysyl-tRNA synthetase activity (e.g., EC:6.1.1.26).
15. The variant or method according any one of the preceding items, wherein: i) said aminoacyl-tRNA synthetase variant (e.g., EC:6.1.1.-.) is a pyrrolysyl-tRNA synthetase variant (e.g., EC:6.1.1.26) and/or ii) said aminoacyl-tRNA synthetase variant exhibits altered properties relative (or compared to) to a corresponding parent pyrrolysyl-tRNA synthetase, e.g., said variant is more efficient than said corresponding parent in AzK incorporation, preferably said variant has an efficiency of AzK incorporation that is at least 10% (e.g., at least 50%, 100%, 150%, 200%, 250%, 300%, 350%, 400%, 450% or 500%) greater than that of said corresponding parent pyrrolysyl-tRNA synthetase (e.g., said variant is capable of yielding the same amount of AzK-labelled polypeptide in a growth medium having only 20% of AzK that is required for the same amount of incorporation by the corresponding parent, e.g., as described in Example 1 herein).
16. A polynucleotide encoding the variant according to any one of the preceding items.
17. A nucleic acid construct comprising the polynucleotide according to any one of the preceding items.
18. An expression vector comprising the polynucleotide and/nucleic acid construct according to any one of the preceding items.
19. A host cell comprising at least one of the following: i) the variant according to any one of the preceding items; ii) the polynucleotide according to any one of the preceding items; iii) the nucleic acid construct according to any one of the preceding items; and/or iv) the expression vector according to any one of the preceding items.
20. The host cell according to any one of the preceding items, wherein said host cell is a recombinant host cell, preferably an isolated recombinant host cell.
21. The host cell according to any one of the preceding items, wherein said host cell is selected from the group consisting of: Methanomethylophilus sp. (e.g., Methanomethylophilus alvus, e.g., Methanomethylophilus alvus Mx1201Ca), E. coli, Corynebacterium glutamicum, Mycoplasma capricolum, CHO, SF9 cells, C. elegans cell, S. cerevisiae, Schizosaccharmyces pombe, Micrococcus luteus, Komagataella pastoris (e.g., Komagataella phaffii), and Bombyx mori.
22. A method for producing the variant according to any one of the preceding items, comprising:
   i) cultivating the host cell according to any one of the preceding items under
   conditions suitable for expression of said variant; and ii) recovering said variant.
23. A composition comprising at least one of the following:
   i) variant according to any one of the preceding items, preferably with a tRNA as orthogonal pair;
   ii) polynucleotide according to any one of the preceding items;
   iii) nucleic acid construct according to any one of the preceding items;
   iv) expression vector according to any one of the preceding items; and/or
   v) host cell according to any one of the preceding items.
24. Use of the variant, polynucleotide, nucleic acid construct, expression vector, host cell or composition according any one of the preceding items, for one or more of the following:
   i) catalyzing the aminoacylation of its cognate tRNA with an aromatic or aliphatic amino acid to form an aminoacyl-tRNA;
   ii) catalyzing the introduction of an aromatic or aliphatic amino acid into a polypeptide;
   iii) site-specific incorporation of non-canonical substrates (e.g., non-canonical amino acids (ncAAs), e.g., with reactive bioorthogonal groups in the side chain, such as an azido-group) into polypeptides, e.g., in response to an amber stop codon.
25. A method for incorporating an aromatic or aliphatic amino acid into a polypeptide, comprising the following steps:
   (i) expressing the polynucleotide, nucleic acid construct or vector according to any one of the preceding items; or providing the variant according to any one of the preceding items and
   (ii) expressing a polynucleotide encoding a tRNA or providing said tRNA, said tRNA and the aminoacyl-tRNA synthetase variant provided in (i) or encoded by the polynucleotide, nucleic acid construct or vector in (i) being an orthogonal pair.
26. A method, system or kit for incorporating an aromatic or aliphatic amino acid into a polypeptide, comprising:
   i) the variant, polynucleotide, nucleic acid construct, expression vector, host cell and/or composition according any one of the preceding items;
   ii) a corresponding amber suppressor tRNA (e.g., orthogonal pair, o-pair, OP).

The invention is further illustrated by the following examples, however, without being limited to the example or by any specific embodiment of the examples.

### EXAMPLES OF THE INVENTION

### Example 1: AzK Incorporation Efficiency of the MaPyIRS M129L+V168C Variant (SEQ ID NO: 2)

Comparing the amino acid binding pockets of the pyrrolysyl-tRNA synthetase enzymes from Candidatus Methanomethylophilus alvus (MaPyIRS) and Methanosarcina mazei (MmPyIRS) it was surprisingly observed that the amino acid sequences in the binding pockets of these two enzymes are rather similar except for a few residues (e.g., Table 1). The orthogonal pair (o-pair) consisting of MmPyIRS and its cognate amber suppressor tRNA, MmtRNA_{CUA}^{Pyl} accepts an extremely diverse set of amino acids, e.g., lysine and pyrrolysine derivatives. Wan (2014), loc. cit. listed over one hundred ncAAs as substrates of wild type and engineered MmPyIRS enzymes (Reference 1). 12 residues, including the selected 6 positions shown in Table 1, in the amino acid binding pockets of the highly homologous PyIRSs from M. mazei and M. barkeri, are most relevant to broaden the substrate scope of engineered PyIRS enzymes (Reference 1).

TABLE 1
Comparison of the selected amino acids in the amino acid
binding pockets of MaPyIRS and MmPyIRS. Differing positions
are indicated in bold print.

| aaRS-position | 1 | 2 | 3 | 4 | 5 | 6 |
|---|---|---|---|---|---|---|
| MaPyIRS | Y126 | M129 | N166 | V168 | Y206 | W239 |
| (SEQ ID NO: 1) | | | | | | |
| MmPyIRS | Y306 | L309 | N346 | C348 | Y384 | W417 |
| (SEQ ID NO: 3) | | | | | | |

The incorporation of different lysine/pyrrolysine derivatives, such as BocK, AllocK and AzK was consequently tested with the wild type MmPyIRS/MmtRNA_{CUA}^{Pyl} and MaPyIRS/MatRNA_{CUA}^{Pyl} o-pairs (e.g., Table 2). An efficient incorporation of AllocK and BocK with both o-pairs was observed. AzK was very well incorporated by the MmPyIRS/MmtRNA_{CUA}^{Pyl} pair while the MaPyIRS/MatRNA_{CUA}^{Pyl} pair was less efficient with this ncAA (e.g., FIG. 1). However, AzK is a particularly useful non-canonical amino acid (ncAA) as it facilitates bioorthogonal conjugation by Cu(I)-catalyzed- or strain-promoted azide-alkyne cycloaddition for the site-selective chemical modification of proteins (Reference 2) Thus, a mutant MaPyIRS with improved acceptance of AzK would be desirable. Consequently, the MaPyIRS amino acid binding pocket mimicking that of MmPyIRS (e.g., Table 1) was engineered.

TABLE 2
Lysine (Lys)/pyrrolysine (Pyl) analogs.

| name | acronym | structure | analog of |
|---|---|---|---|
| N^{ϵ}-((2- azidoethoxy)carbonyl)- L-lysine | AzK | 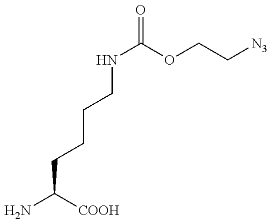 [SMILES: [N-]=[N+]=NCCOC(=O)NCCCC[C@H](N)C(=O)O] | Lys/Pyl |
| N^{ϵ}-allyloxycarbonyl-L- lysine | AllocK | 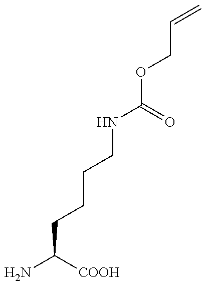 [SMILES: C=CCOC(=O)NCCCC[C@H](N)C(=O)O] | Lys/Pyl |
| N^{ϵ}-(tert- butyloxycarbonyl)-L- lysine | BocK | 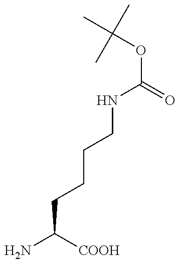 [SMILES: CC(C)(C)OC(=O)NCCCC[C@H](N)C(=O)O] | Lys/Pyl |

The MaPyIRS M129L+V168C mutant variant was generated, which contains the same amino acid residues in the amino acid binding pocket as the MmPyIRS. MaPyIRS M129 corresponds to MmPyIRS residue L309 and MaPyIRS V168 corresponds to C348 of MmPyIRS, while the other four relevant amino acids are identical in both enzymes (e.g., Table 1). The mutant o-pair MaPyIRS M129L V168C/MatRNA_{CUA}^{Pyl} was tested for the incorporation of AzK in comparison to the wild type MmPyIRS/MmtRNA_{CUA}^{Pyl} and MaPyIRS/MatRNA_{CUA}^{Pyl} pairs. The efficiency of the incorporation of AzK into the reporter protein eGFP Y40am was assessed by recording the fluorescence intensity normalized to the corresponding cell density and was compared to the fluorescence of wild type eGFP expressed from the same plasmid.

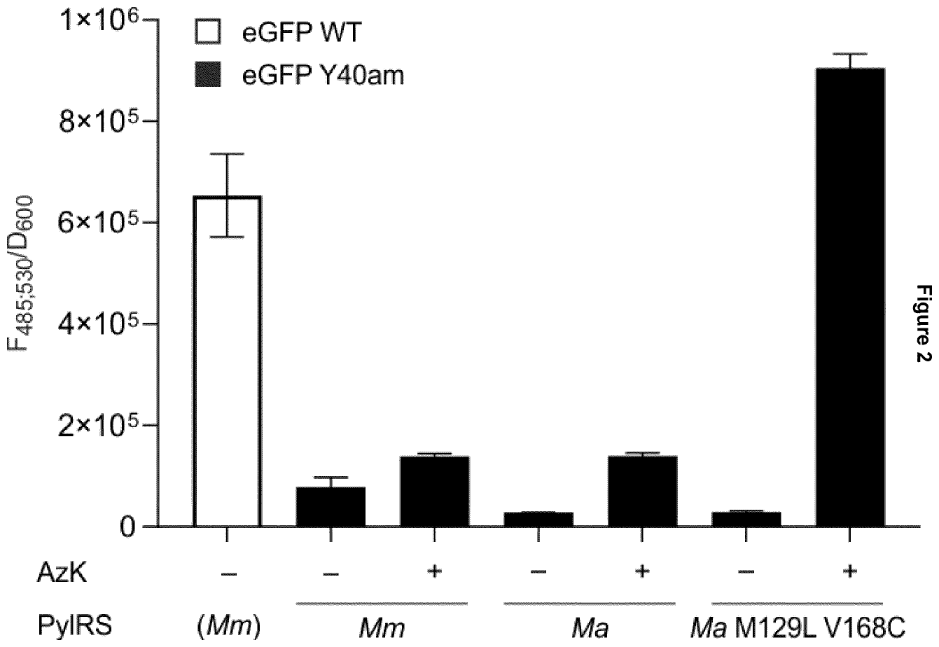

The MaPyIRS M129L V168C/MatRNA_{CUA}^{Pyl} showed markedly improved AzK incorporation in comparison to the wild type MaOP (e.g., FIG. 2). Surprisingly, the efficiency was so high that the fluorescence signal of the reporter protein eGFP Y40AzK exceeded that of the wild type eGFP. Moreover, analogous to the wild type, the mutant pair showed excellent orthogonality towards canonical amino acids since the eGFP fluorescence was negligible in the absence of an ncAA. Again, the mutant MaOP performed better in this regard than the wild type MmOP that was used as a benchmark. The results confirmed that the substrate tolerance of the MaPyIRS enzyme could be broadened through the co-called “transfer” of amino acid binding pocket residues of the MmPyIRS enzyme.

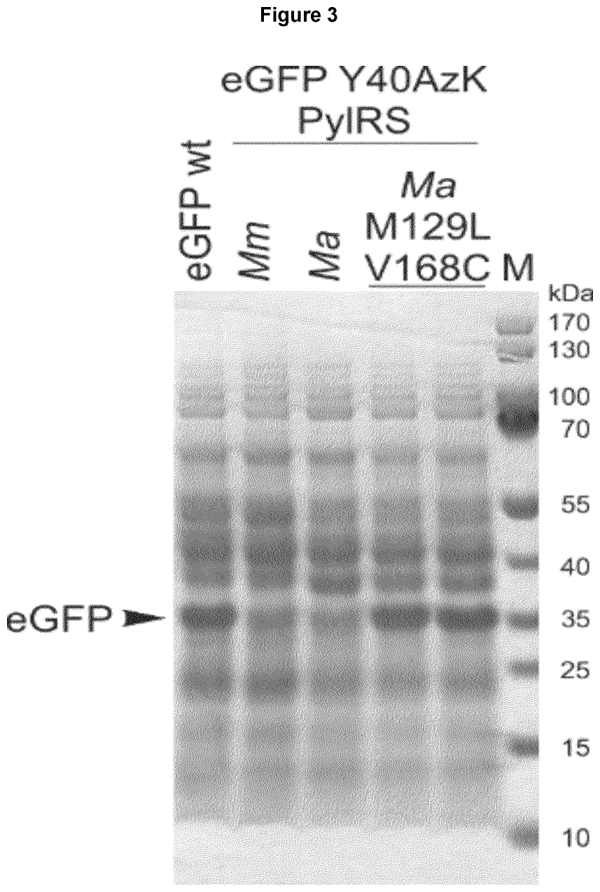

To further validate the finding, the expression of the reporter protein eGFP by SDS-PAGE was analyzed (e.g., FIG. 3). In agreement with the fluorescence measurements, it was observed that eGFP expression band in the presence of AzK with the MaPyIRS M129L V168C/MatRNA_{CUA}^{Pyl} o-pair was comparable to the intensity of the eGFP wild type band. In contrast, the MmOP and wild type MaOP produced a markedly less intense eGFP band in the presence of AzK.

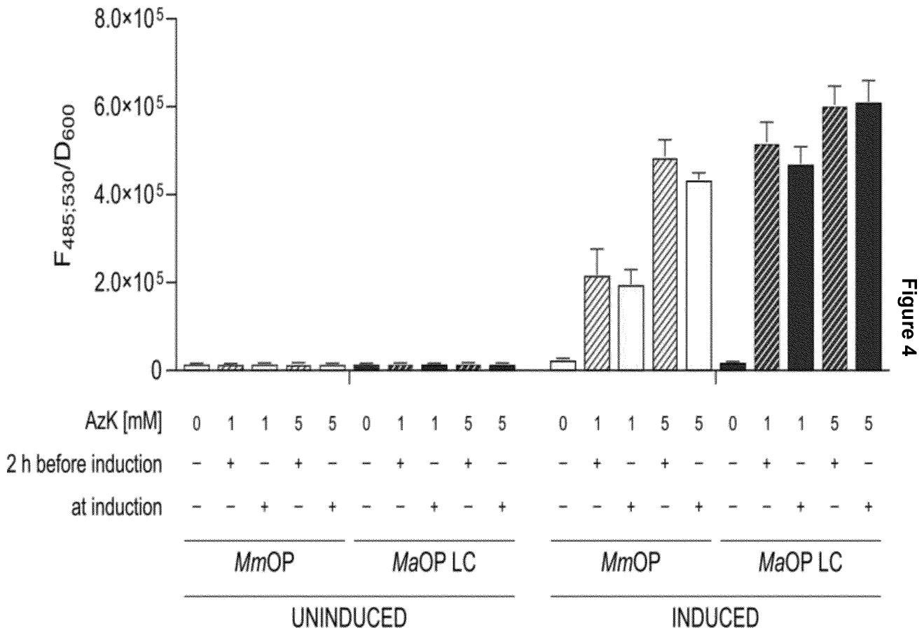

To our surprise, the mutant MaOP LC comprised of MaPyIRS M129L V168C/MatRNA_{CUA}^{Pyl} used AzK more efficiently than the benchmark wild-type MmOP. The expression level of eGFP Y40AzK was similar with 1 mM and 5 mM AzK. Moreover, at supplementation with 1 mM AzK, MaOP LC produced 2.4-fold the level of eGFP Y40AzK as MmOP. The difference was less pronounced with 5 mM AzK, but still the level was higher with MaOP LC (1.2-fold if AzK was added before the induction and 1.4-fold when AzK and IPTG were added in parallel). Supplementation of the medium with AzK 2 hours before the induction of the target protein yielded slightly more target protein with both o-pairs, which hints at a better intracellular accumulation of the ncAA (e.g., FIG. 4). Consequently, the application of the mutant MaOP could yield the same amount of AzK-labeled target protein by supplying only a fifth of the AzK in the medium, and four fifths of the cost for AzK might be saved. This observation is particularly important for up-scaling.

### REFERENCES

1. Wan W, Tharp J M, Liu W R. 2014. Pyrrolysyl-tRNA synthetase: An ordinary enzyme but an outstanding genetic code expansion tool. Biochimica et Biophysica Acta (BBA)—Proteins and Proteomics 1844(6): 1059-1070.
2. Li L, Zhang Z. 2016. Development and applications of the copper-catalyzed azide-alkyne cycloaddition (CuAAC) as a bioorthogonal reaction. Molecules 21(10): 1393.

1. A variant of a parent aminoacyl-tRNA synthetase (e.g., EC:6.1.1.-.), wherein said variant comprises:
i) a substitution at one or more positions corresponding to positions 168 and 129 of the amino acid sequence set forth in SEQ ID NO: 1 (e.g., MaPyIRS), (e.g., using the numbering of SEQ ID NO: 1), wherein said variant comprises the following combination of substitutions: X168C+X129L (e.g., V168C+M129L)
ii) said variant is a polypeptide having at least 70%, e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, but less than 100% sequence identity with the amino acid sequence set forth in SEQ ID NO: 1 (e.g., MaPyIRS),
iii) said variant has aminoacyl-tRNA synthetase activity (e.g., EC:6.1.1.-.).

2. A method for obtaining an aminoacyl-tRNA synthetase variant (e.g., EC:6.1.1.-.), said method comprising:
i) introducing into a parent aminoacyl-tRNA synthetase a substitution at one or more positions corresponding to positions 168 and 129 of the polypeptide of SEQ ID NO: 1 (e.g., MaPyIRS), (e.g., using the numbering of SEQ ID NO: 1), wherein said variant has aminoacyl-tRNA synthetase activity (e.g., EC:6.1.1.-.), wherein said variant comprises the following combination of substitutions: X168C+X129L (e.g., V168C+M129L)
ii) recovering said variant.

3. The variant or method according any one of the preceding claims, wherein said variant comprises the following combination of substitutions: V168C+M129L, corresponding to positions of the amino acid sequence as set forth in SEQ ID NO: 1 (e.g., using the numbering of SEQ ID NO: 1).

4. The variant or method according any one of the preceding claims, wherein said variant is a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, or 100% sequence identity with the amino acid sequence as set forth in SEQ ID NO: 2 (e.g., MaPyIRS V168C+M129L).

5. The variant or method according any one of the preceding claims, wherein said variant comprises or consists of SEQ ID NO: 2.

6. The variant or method according any one of the preceding claims, wherein said variant is capable of catalyzing the aminoacylation of its cognate tRNA with an aromatic or aliphatic amino acid to form an aminoacyl-tRNA.

7. The variant or method according any one of the preceding claims, wherein said amino acid is a lysine derivative, preferably said variant comprises a combination of substitutions X168C+X129L (e.g., V168C and M129L).

8. The variant or method according any one of the preceding claims, wherein said lysine derivative is selected from the group consisting of: N^{ε}-((2-azidoethoxy)carbonyl)-L-lysine (AzK), pyrrolysine, boc-lysine, alloc-lysine, azide-lysine, 2-N,6-N-Bis(2,3-dihydroxy-N-benzoyl)-L-serine, 2-N,6-N-Bis(2,3-dihydroxy-N-benzoyl)-L-serine amide, 3-hydroxylysine, N-benzoylglycyl-N⁶-[2-hydroxy-2-(3-methylquinoxalin-2-yl)ethyl]lysine, N-benzoylglycyl-N⁶-[2-hydroxy-3-(quinoxalin-2-yl)propyl]lysine, N-hippuryl-N⁶-(carboxymethyl)lysine, N⁶-(2,4-dinitrophenyl)lysine, N⁶-(2-carboxyethyl)lysine, N-acetonyllysine, N⁶-carbamoylmethyllysine, N⁶-methyllysine, hydroxylysine, isodesmosine, ornithine derivatives such as 2-amino-5-(prop-2-ynoylamino)pentanoic acid (5-(prop-2-ynoylamino)ornithine), and 2-amino-5-[(azidoacetyl)amino]pentanoic acid.

9. The variant or method according any one of the preceding claims, wherein said lysine derivative is N^{ε}-((2-azidoethoxy)carbonyl)-L-lysine (AzK), preferably said variant comprises a combination of substitutions X168C+X129L (e.g., V168C and M129L).

10. The variant or method according any one of the preceding claims, wherein said variant is capable of accepting non-canonical substrates, preferably non-canonical amino acids (ncAAs) (e.g., ncAAs with reactive bioorthogonal groups in the side chain, e.g., an azido-group).

11. The variant or method according any one of the preceding claims, wherein said parent aminoacyl-tRNA synthetase (e.g., EC:6.1.1.-.) is obtainable from a Methanomethylophilus sp. (e.g., Methanomethylophilus alvus, e.g., Methanomethylophilus alvus Mx1201Ca).

12. The variant or method according any one of the preceding claims, wherein said aminoacyl-tRNA synthetase activity (e.g., EC:6.1.1.-.) comprises a pyrrolysyl-tRNA synthetase activity (e.g., EC:6.1.1.26).

13. The variant or method according any one of the preceding claims, wherein said aminoacyl-tRNA synthetase variant (e.g., EC:6.1.1.-.) is a pyrrolysyl-tRNA synthetase variant (e.g., EC:6.1.1.26).

14. A polynucleotide encoding the variant according to any one of the preceding claims.

15. A nucleic acid construct comprising the polynucleotide according to any one of the preceding claims.

16. An expression vector comprising the polynucleotide and/nucleic acid construct according to any one of the preceding claims.

17. A host cell comprising at least one of the following: i) the variant according to any one of the preceding claims; ii) the polynucleotide according to any one of the preceding claims; iii) the nucleic acid construct according to any one of the preceding claims; and/or iv) the expression vector according to any one of the preceding claims.

18. The host cell according to any one of the preceding claims, wherein said host cell is a recombinant host cell, preferably an isolated recombinant host cell.

19. The host cell according to any one of the preceding claims, wherein said host cell is selected from the group consisting of: Methanomethylophilus sp. (e.g., Methanomethylophilus alvus, e.g., Methanomethylophilus alvus Mx1201Ca), E. coli, Corynebacterium glutamicum, Mycoplasma capricolum, CHO, SF9 cells, C. elegans cell, S. cerevisiae, Schizosaccharmyces pombe, Micrococcus luteus, Komagataella pastoris (e.g., Komagataella phaffii), and Bombyx mori.

20. A method for producing the variant according to any one of the preceding claims, comprising:
i) cultivating the host cell according to any one of the preceding claims under conditions suitable for expression of said variant; and
ii) recovering said variant.

21. A composition comprising at least one of the following:
i) variant according to any one of the preceding claims, preferably with a tRNA as orthogonal pair;
ii) polynucleotide according to any one of the preceding claims;
iii) nucleic acid construct according to any one of the preceding claims;
iv) expression vector according to any one of the preceding claims; and/or
v) host cell according to any one of the preceding claims.

22. Use of the variant, polynucleotide, nucleic acid construct, expression vector, host cell or composition according any one of the preceding claims, for one or more of the following:
i) catalyzing the aminoacylation of its cognate tRNA with an aromatic or aliphatic amino acid to form an aminoacyl-tRNA;
ii) catalyzing the introduction of an aromatic or aliphatic amino acid into a polypeptide;
iii) site-specific incorporation of non-canonical substrates (e.g., non-canonical amino acids (ncAAs), e.g., with reactive bioorthogonal groups in the side chain, such as an azido-group) into polypeptides, e.g., in response to an amber stop codon.

23. A method for incorporating an aromatic or aliphatic amino acid into a polypeptide, comprising the following steps:
(i) expressing the polynucleotide, nucleic acid construct or vector according to any one of the preceding claims; or providing the variant according to any one of the preceding claims and
(ii) expressing a polynucleotide encoding a tRNA or providing said tRNA, said tRNA and the aminoacyl-tRNA synthetase variant provided in (i) or encoded by the polynucleotide, nucleic acid construct or vector in (i) being an orthogonal pair.

24. A method, system or kit for incorporating an aromatic or aliphatic amino acid into a polypeptide, comprising:
i) the variant, polynucleotide, nucleic acid construct, expression vector, host cell and/or composition according any one of the preceding claims;
ii) a corresponding amber suppressor tRNA (e.g., orthogonal pair, o-pair, OP).
